SEQUENCE LISTING (25 sequences)

SEQ ID NO: 1 - DNA, 5489 bp, Homo sapiens
  misc_feature (1)..(5489): gi|187608552|ref|NM_004354.2| (CyclinG2)
gaaactcttaacaaaaacaaggggctcggggaggtttccgctgaggcggcgggggtgcggcggtgggctggtcttccgcggccggcgttgcgccgcggcggagggtgggcgcgcggggagcgggatggagccggggctgtgaggccgaggcggcggtgcctgggaggaagggtcggatgccggaccgggggcaccgctgaggcggtgggtccccgacctgcgagacaggtttggaagcccccgctgcgcccagtccgtgcggaccgcgaggccgcgggcgggtggaggcgcgtctccggcacgatgaaggatttgggggcagagcacttggcaggtcatgaaggggtccaacttctcgggttgttgaacgtctacctggaacaagaagagagattccaacctcgagaaaaagggctgagtttgattgaggctaccccggagaatgataacactttgtgtccaggattgagaaatgccaaagttgaagatttaaggagtttagccaacttttttggatcttgcactgaaacttttgtcctggctgtcaatattttggacaggttcttggctcttatgaaggtgaaacctaaacatttgtcttgcattggagtctgttcttttttgctggctgctagaatagttgaagaagactgcaatattccatccactcatgatgtgatccggattagtcagtgtaaatgtactgcttctgacataaaacggatggaaaaaataatttcagaaaaattgcactatgaattggaagctactactgccttaaactttttgcacttataccatactattatactttgtcatacttcagaaaggaaagaaatactgagccttgataaactagaagctcagctgaaagcttgcaactgccgactcatcttttcaaaagcaaaaccatctgtattagccttgtgccttctcaatttggaagtggaaactttgaaatctgttgaattactggaaattctcttgctagttaaaaaacattccaagattaatgacactgagttcttctactggagagagttggtttctaaatgcctagccgagtattcttctcctgaatgttgcaaaccagatcttaagaagttggtttggatcgtttcaaggcgcacagcccagaacctccacaacagctactatagtgttcctgagctgccaacgatacctgaggggggttgttttgatgaaagtgaaagtgaggactcttgtgaagatatgagttgtggagaggagagtctcagcagctctcctcccagtgatcaagagtgcaccttctttttcaacttcaaagtggcacaaacactgtgctttccatcttagaaatctgattgttctgtcagaatttatatttacaggtttcaaagcaataaatgggggaataggtagtttcctggtttagcccccatctagtcaggaattaatatactggaatacctaccttctatttgttattcagatcagatctggcctattttcatatttatcctaagccatcaaatggggtagtgcctcttaaaccattaacagtactttagacattggcactttatttttctcgtagatctttagctactttggggaggagggaaggtgctgataccttcaatttgttacttttcaagatttttaaaaataactagtgtagcttatcttaaacattttataaaaccttcagatgtctttaagcagattggaagtatgcaagtgcttccttagcagggacagtggataatccttaatggtttatcatagatttcaccctccccccttctcagaagagtgagtatgctcttaaatgtcaaacacatttttgttgttttgttttttaaatgatcagtgtctatttgatgtgatgcagatcttataaatttgggaattataatattgacatttctgtgatttttatatatgtaatgtcttaattgagatttctgttaaggcagaaataattaggctagggctcttagttttcattcctattgcccaagtattgtcaaactatggtattattttaatgttactttaaaaatccataatctgctagttttgcatgtacttatatgaaaacagtgcagtaagttgaaaactcagtatctatggaattgataaatgttgatctggtgtagtatattttatcgcattttcttatattaaaaaatgtctgcatgattacattttatttcctttgtaatttacatttcagaatagtgtattgctatatgggtgccaagattgaatatgaagaacccgagtgtttgtagtattatagttttaagcaaatctgtgtggtgatacagccataagaatggggcttatataaactctgtacatgtaagattttgtacagagaatttttaactttataaattgtatatgaacatgtaaatcttttaaaatgtacataaaatactgtatttttttaccttgtgtgtgatagtctagtcattgcatgtaaatataatttattatgtattctgtagtataaatcatacattgatgacttacatttttactggtaagtcaacatccgttggatgttttctgaagtggctctttttgaagtgataatagattgtaattcaaaataaaattattaatgaattctccttgtttgggatcacatcttaatttttaatctgttaaaagttcttgatgtattttaatgagaagactttaggtgaggctacagtgattccagagtgagccttctaactggctagcagaagttctctaggtttggcatctgtgccttggagatactgaaagagaatctgtcatttgacaattgacctctttgtgggatggactcattaagtatgctctcagagactggtatattaccagaatgcctattaattttcagtgagaggcaacaggtattaagtagaacagaatgctcaggttggcagattagaacgatctttcaggagacaaagcaagttttaatcagttgtttggttaataagtatggggtgttcgctgtgatagggccccgccagcttctggctcttgtggacctcaaaagtatcaggtggttttgcaagtggtggtcctttcccctgccccaccccaataggttccccatctgtctagtttgatttttgtagacctttgttttctctagttagaaaatcaggtacactgaatatggttttcatgtaacacctcttctctggagataggggtatgttttcctacccttctagtggagaatcctacttgaggatgacctttcctctcttactaaataatattagtaaatagtgggcaatatattctgctttcagattttgatttgttgagatgtaaaagttgtttggggcttaccaaatctcaagactctctttagctcctgcaggattgtattgcttttcttactggatatttttcctgggtaagcatctttgtggcttcatctcttccccctgtggttttcagtgtatttagtcgagacctctctgctgagcttgcaacctgtttattcacatggcctgccatgccacttggaggtttctgattactcccaaacctgctggttctttatgtctttctcagcgaataattccatctattcatgttggaaacttaggtgatatgctcatctccttttgcctgtttatggaggtcaccagcctctatcatttgtatgatttcgtttacactgtttatatctctctgtcccccctttttctgccattggcatggtttagacctgtactctttatcagcagaggtactgtaatatatttgtgatccctcagcttccaggcttactcctggtctctgccttcctatctacatatccttttaaaataaaattttaactatctcctgaaaaattgttgagtaggtcacgcacaatcaggagaaaaatctattcatgacatacaagtctctgtctaatctgaacactgcacctgtctctggcctttttttcttgtcatttcctagaccttaaaaaatgtgtattgagaaagaactctgttagctatacagaagatgaactgggcaatatagagtagcagcatggagaccagtctgactgaactaaggcagtggaagtgtggatgaggaagagaggtgaaaattgagaagcgctatcctttctctttgggcattattaggaggctcacagacaagtccaggagcctggttataccctcctgtgccattcaaccaggtggctttcccatgactgtgatgaataaaattgagaagcccctgcccttttcagagcagagggtgaggagaaagctaccattttgtcctcatccttacccccgttgacttggcgagagatttgacctttcaggttttgatcctgtcattttctaggatgtggtgcacgcactttgctgttgcgcatggtgaagtattgtgcctaggtcctgggtcttcatctgtttggctctgctactgtttcctcctcccaggaagtgtggttagacaaataatgtgttttaattacctgtcacactcaggattaatacatactcaggttaactgtagagaggcattggcttcagaacactcctcgtgacaattttaaccattttctttgtctagagtctgcctttttcttttttacaatttcttttatttcaacactaggtttcaatatggtgttcctgctacctcccacctccctcctccctcatcacacatgcaaattgtcagcttattgagacaacccacttagattcatatatggacaaggacaaggtattttgcatttgttactggaattcagttttcctaactatttactaccagaaatggtcaataacttactttgtgtttagcaaatcaaattgtgtgatagatagtttcccagtatgatggccagtcagtctttccatccctgtgcctacatgctgctcttcccgtccacaagtggagtctgtttctcttgagttttggctggccttatgaatggctttgcttactgaagtgcagcagaagaaatttagtatatgtccaagcctaggctttaagagactggcagctttccttttatcctttttggaagctagccaccatgctgcaaagaagctcagctggattactgaaagatgagaggccatgtggagagagactcttgaggatgagagattatcttggatgttccagccttaagctcccagctgaatgtgggtgtatcctcagctacaccacagaaaacagaggaactactcagtcgatcccaatcaacccacagactcactagaaataacaaattattgttttaagccacgaggttttgggggagggttgttaaacagtaatagataagtgagacagattgcttgttatttatggtcaaatggtgattatctctggtgagattacaggtgatgttttttttaagttatgcctatctgtagtttcctttttttcctaaaattgatttgaattattagtgtattaacagaataaagaatgaactttaaaacacaaaaaaaaaaaaaaaa

SEQ ID NO: 2 - DNA, 3796 bp, Homo sapiens
  misc_feature (1)..(3796): gi|209529713|ref|NM_030762.2| (Sharp1)
ctggtctggccggcgacgcgcgtgccctgtggccaaacactgcctggagtgagagcaaactaccagcgcagtggggccggcgcgagtgtgcgtgtgtgtgcgtgtgtgtgtgcgagcgcggtggaggggggggaccaactgcttcacactttcaacactgcactgaagagggagagcgagagagagactggagacgcacagatccccccaaggtctcccaagcctaccgtcccacagattattgtacagagccccaaaaatcgaaacagaggaaacgaacagcagttgaacatggacgaaggaattcctcatttgcaagagagacagttactggaacatagagattttataggactggactattcctctttgtatatgtgtaaacccaaaaggagcatgaaacgagacgacaccaaggatacctacaaattaccgcacagattaatagaaaagaaaagaagagaccgaattaatgaatgcattgctcagctgaaagatttactgcctgaacatctgaaattgacaactctgggacatctggagaaagctgtagtcttggaattaactttgaaacacttaaaagctttaaccgccttaaccgagcaacagcatcagaagataattgctttacagaatggggagcgatctctgaaatcgcccattcagtccgacttggatgcgttccactcgggatttcaaacatgcgccaaagaagtcttgcaatacctctcccggtttgagagctggacacccagggagccgcggtgtgtccagctgatcaaccacttgcacgccgtggccacccagttcttgcccaccccgcagctgttgactcaacaggtccctctgagcaaaggcaccggcgctccctcggccgccgggtccgcggccgccccctgcctggagcgcgcggggcagaagctggagcccctcgcctactgcgtgcccgtcatccagcggactcagcccagcgccgagctcgccgccgagaacgacacggacaccgacagcggctacggcggcgaagccgaggcccggccggaccgcgagaaaggcaaaggcgcgggggcgagccgcgtcaccatcaagcaggagcctcccggggaggactcgccggcgcccaagaggatgaagctggattcccgcggcggcggcagcggcggcggcccggggggcggcgcggcggcggcggcagccgcgcttctggggcccgaccctgccgccgcggccgcgctgctgagacccgacgccgccctgctcagctcgctggtggcgttcggcggaggcggaggcgcgcccttcccgcagcccgcggccgccgcggcccccttctgcctgcccttctgcttcctctcgccttctgcagctgccgcctacgtgcagcccttcctggacaagagcggcctggagaagtatctgtacccggcggcggctgccgccccgttcccgctgctataccccggcatccccgccccggcggcagccgcggcagccgccgccgccgctgccgccgccgccgccgcgttcccctgcctgtcctcggtgttgtcgccccctcccgagaaggcgggcgccgccgccgcgaccctcctgccgcacgaggtggcgccccttggggcgccgcacccccagcacccgcacggccgcacccacctgcccttcgccgggccccgcgagccggggaacccggagagctctgctcaggaagatccctcgcagccaggaaaggaagctccctgaatccttgcgtcccgaaggacggaggttcaagcagagtgagaagttaaaatacccttaaggaggttcaagcagagtgagaagttaaaatacccttaaggtctttaagggaggaagtgtaatagatgcacgacaggcataaacaagaacaacaaaacaggtgttatgtgtacattcggagttcctgttttgctcatcccgcaccaccccaccctccacacactaacatccctttcttccccccaccagctgtaaaagatcctatgcgaaagacactggctcttttttttaatcccccaaataaattttgcccccttttaggccatgttccattatctcttaaaattggaacctaattcgagaggaagtaagaagggtctgttctgtggctgagctaggtgaaccccggggtaggggaaagatgttaacacctttgacgtctttggagttgacatggaacagcaggtagttgttatgtagagctagttctcaaagctgccctgcctgttttaggaggcgttccacaaacagattgaggctcttttagaattgaatttactcttcagtattttctaatgttcagctttctaaaaggcatatatttttcaaagaagtgaggatgcagtttctcacgttgcaacctattctgaagtggtttaaatggtatctcttagtaacttgcactcgttaaagaaacacggagctgggccatcgtcagaactaagtcagggaaggagatggatgagaaggccagaatcattcctagtacatttgctaacactttattgagaaattgaccatgaattaatggactcatcttaatttcttctaagtccatatatagatagatatctatctgtacagatttctatttatccatagataggtatctatacatacacatctcaagtgcatctattcccactctcattaatccatcatgttcctaaatttttgtaatcttactgtaaaaaaaagtgcactgaacttcaaaacaaaacaaaaaacaacaacaacaaaaaacaagtccaaactgatatatcctatattctgttaaaattcaaaagtgaacgaaagcatttaactggccagttttgattgcaaatgctgtaaagatatagaatgaagtcctgtgaggccttcctatctccaagtctatgtattttctggagaccaaaccagataccagataatcacaaagaaagcttttttaataaggcttaaaccaagaccttgtctagatatttttagtttgttgccaaggtagcactgtgagaaatctcacttggatgttatgtaaggggtgagacacaacagtctgactatgagtgaggaaaatatctgggtcttttcgtcagtttggtgcatttgctgctgctgttgctactgtttgcctcaaacgctgtgtttaaacaacgttaaactcttagcctacaaggtggctcttatgtacatagttgttaatacatccaattaatgatgtctgacatgctatttttgtagggagaaaatatgtgctaatgatattttgagttaaaatatcttttggggaggatttgctgaaaagttgcacttttgttacaatgcttatgcttggtacaagcttatgctgtcttaaattattttaaaaaaataaatactgtctgtgagaaaccagctggtttagaaaagtttagtatgtgacgataaactagaaattacctttatattctagtattttcagcactccataaattctattacctaaatattgccacactattttgtgatttaaaaattcttactaaggaataaaaactttaatatacgatatgatattgtctaataattaaaaaagacataatggatgctcaattagttttaagatatctataactatagggatacaaatcactacagttctcagatttacagcttttttttgtcattggcttgatgtcacacatttccaatctcttgcaagcctccaggctctggctttgtctacctgctcgttcccaatgtatcttaatgaaaagtgcaaaagaaaaacctaccaattaaaaaaaaaaaaaaaaaa

SEQ ID NO: 3 - DNA, 23 bp, Homo sapiens
  misc_feature (1)..(12): Actin for
atgaagtgtgacgttgacatccg

SEQ ID NO: 4 - DNA, 22 bp, Homo sapiens
  misc_feature (1)..(22): Actin rev
gcttgctgatccacatctgctg

SEQ ID NO: 5 - DNA, 22 bp, Homo sapiens
  misc_feature (1)..(22): p53 for
ctggcccctgtcatcttctgtc

SEQ ID NO: 6 - DNA, 22 bp, Homo sapiens
  misc_feature (1)..(22): p53 rev
cacgcaaatttccttccactcg

SEQ ID NO: 7 - DNA, 20 bp, Homo sapiens
  misc_feature (1)..(20): SHARP1
gcatgaaacgagacgacacc

SEQ ID NO: 8 - DNA, 20 bp, Homo sapiens
  misc_feature (1)..(20): SHARP1 rev
cgctccccattctgtaaagc

SEQ ID NO: 9 - DNA, 21 bp, Homo sapiens
  misc_feature (1)..(21): CyclinG2 for
cctcccagtgatcaagagtgc

SEQ ID NO: 10 - DNA, 20 bp, Homo sapiens
  misc_feature (1)..(20): CyclinG2 rev
tccctcctccccaaagtagc

SEQ ID NO: 11 - DNA, 19 bp, Homo sapiens
  misc_feature (1)..(19): GAPDH for
agccacatcgctcagacac

SEQ ID NO: 12 - DNA, 19 bp, Homo sapiens
  misc_feature (1)..(19): GAPDH rev
gcccaatacgaccaaatcc

SEQ ID NO: 13 - DNA, 20 bp, Homo sapiens
  misc_feature (1)..(20): SHARP1 for
cgtctttggagttgacatgg

SEQ ID NO: 14 - DNA, 20 bp, Homo sapiens
  misc_feature (1)..(20): SHARP1 rev
gggcagctttgagaactagc

SEQ ID NO: 15 - DNA, 20 bp, Homo sapiens
  misc_feature (1)..(20): CyclinG2 for
tggacaggttcttggctctt

SEQ ID NO: 16 - DNA, 24 bp, Homo sapiens
  misc_feature (1)..(24): CyclinG2 rev
gatggaatattgcagtcttcttca

SEQ ID NO: 17 - DNA, 19 bp, Homo sapiens
  misc_feature: GFP
caagctgaccctgaagttc

SEQ ID NO: 18 - DNA, 19 bp, Homo sapiens
  misc_feature (1)..(19): Human p53
gactccagtggtaatctac

SEQ ID NO: 19 - DNA, 20 bp, Homo sapiens
  misc_feature (1)..(20): p53
ccgcgccatggccatctaca

SEQ ID NO: 20 - DNA, 19 bp, Homo sapiens
  misc_feature (1)..(19): Smad4
gtacttcataccatgccga

SEQ ID NO: 21 - DNA, 19 bp, Homo sapiens
  misc_feature (1)..(19): Sharp1 A
gctttaaccgccttaaccg

SEQ ID NO: 22 - DNA, 19 bp, Homo sapiens
  misc_feature (1)..(19): Sharp1 B
cgagacgacaccaaggata

SEQ ID NO: 23 - DNA, 19 bp, Homo sapiens
  misc_feature (1)..(19): CyclinG2 A
gagtcggcagttgcaagct

SEQ ID NO: 24 - DNA, 19 bp, Homo sapiens
  misc_feature (1)..(19): CyclinG2 B
agaatactcggctaggcat

SEQ ID NO: 25 - DNA, 19 bp, Homo sapiens
  misc_feature (1)..(19): Control
ttctccgaacgtgtcacgt

(19) United States
(12) Patent Application Publication
(10) Pub. No.: US 2012/0035069 A1
(43) Pub. Date: Feb. 9, 2012

(54) PROGNOSIS OF BREAST CANCER PATIENTS BY MONITORING THE EXPRESSION OF TWO GENES

(76) Inventors: Stefano Piccolo, Padova (IT); Michelangelo Cordenonsi, Padova (IT); Maddalena Adorno, Monte San Pietro (IT); Silvio Bicciato, Noventa Padovana (IT)

(21) Appl. No.: 13/145,640

(22) PCT Filed: Jan. 21, 2009
(86) PCT No.: PCT/EP2009/050643

§ 371 Date: Jul. 21, 2011

(51) Int. Cl. C12Q 1/68; G01N 21/76; C40B 30/04; G01N 33/566

(52) U.S. Cl. 506/9; 435/7.9; 436/501; 435/6.12

(57) ABSTRACT

The present invention relates to the expression of two genes, CyclinG2 and Sharp1, which correlates with prognosis in individuals having breast cancer. Specifically, this invention provides a method to stratify samples from breast cancer patients in a high or low recurrence risk in the years following primary tumor removal. This classification can be achieved through the analysis of protein or mRNA expression levels for the two identified genes. The invention also illustrates how CyclinG2 and Sharp1 have been identified in mammary cancer cell lines and validated in a large cohort of human patients as powerful metastasis predictors.

### FIELD OF THE INVENTION

The present invention is related to a minimal gene signature providing useful information by molecular methods based on nucleic acid or on protein levels on breast cancer recurrence.

### BACKGROUND ART

Breast cancer is the most common cancer in women. In the US, 1 in 8 women are expected to develop some type of breast cancer by age 85.

While mechanism of tumorigenesis for most breast carcinomas is largely unknown, there are genetic factors that can predispose some women to developing breast cancer (Miki et al., 1994). The discovery and characterization of BRCA1 and BRCA2 has recently expanded our knowledge of genetic factors which can contribute to familial breast cancer although only about 5% to 10% of is breast cancers are associated with BRCA1 and BRCA2. BRCA1 is a tumor suppressor gene that is involved in DNA repair and cell cycle control, which are both important for the maintenance of genomic stability. Like BRCA1, BRCA2 is involved in the development of breast cancer and plays a role in DNA repair, while, unlike BRCA1, it is not involved in ovarian cancer.

Other genes have been linked to breast cancer, for example c-erb-2 (HER2) and p53 (Beenken et al., 2001). Overexpression of c-erb-2 (HER2) and p53 have been correlated with poor prognosis.

However to date, no other clinically useful markers consistently associated with breast cancer have been identified for sporadic tumors, i.e. those not currently associated with a known germline mutation, which constitute the majority of breast cancers.

In clinical practice, accurate diagnosis of various subtypes of breast cancer is important because treatment options, prognosis, and the likelihood of therapeutic response all vary broadly depending on the diagnosis. Early diagnosis and risk stratification is extremely important in this cancer, as breast cancer morbidity and mortality increases significantly if detection occurs late during its progression.

Accurate prognosis or determination of distant metastasis-free survival could allow the oncologist to tailor the administration of adjuvant chemotherapy, with women having poorer prognoses being given the most aggressive treatment. Furthermore, accurate prediction of poor prognosis would greatly impact clinical trials for new breast cancer therapies, because potential study patients could then be stratified according to prognosis.

Typically, the diagnosis of breast cancer requires histopathological proof of the presence of the tumor. In addition to diagnosis, histopathological examinations also provide information about prognosis and selection of treatment regimens. Prognosis may also be established based upon clinical parameters such as tumor size, tumor grade, the age of the patient, and lymph node colonization by tumor cells.

Diagnosis and/or prognosis may be determined to varying degrees of effectiveness by direct examination of the outside of the breast, or through mammography or other X-ray imaging methods. The latter approach is not without is considerable social and personal costs, however.

Recently, the FDA has approved MammaPrint®, a gene expression profiling test system for breast cancer prognosis, based on cDNA microarray analysis for more than 70 genes, determined in fresh or frozen breast cancer biopsies, based on the study of van't Veer, published in (van't Veer et al., 2002).

Even though this test is for physicians’ use only, it has nevertheless to be carried out on special instrumentation, such as a DNA Bioanalyzer/microarray scanner.

This represents a major drawback, since the result can only be provided by large hospitals or companies who developed means and standard procedures to carry out such a complex analysis.

From the above, the advantages of the present invention based on the predictive prognostic value of the analysis of the expression of only two genes, can be easily understood.

The simultaneous analysis of tens of genes requires indeed the array technology, which is instead not necessary for the simple evaluation of expression of CyclinG2 (CCNG2) and Sharp1 (BHLHB3, BHLHE41). From the other side, standard methods for breast cancer prognosis, like the evaluation of the primary mass, lymph node involvement and staging of the cancer, are nowadays insufficient to predict the progression of the disease. Coupling traditional histological methods with a molecular characterization of the tumor through this minimal signature will allow a fine and inexpensive way to predict the course of the disease and the risk of recurrence, especially for cancers defined as medium-aggressive with canonical criteria.

### SUMMARY OF THE INVENTION

The invention is related to a method for evaluating a breast cancer patient's risk of recurrence comprising detecting the level of CyclinG2 (Gene ID=901) gene expression alone or in combination with Sharp1 (Gene ID=79365) in a sample. The detection comprises measuring a signal directly related to the gene(s) expression in said sample, acquiring the signal and evaluating the risk of cancer recurrence of a breast cancer patient by:
calculating a signature score for CyclinG2 gene expression values alone or for, preferably, both CyclinG2 and Sharp1 expression values in the is unknown sample, wherein said signature score is defined as:

$${\sum }_{k = 1}^{K} \frac{{x}_{i}^{k} - {\hat{μ}}^{k}}{{\hat{σ}}^{k}}$$

being K=1 when using CyclinG2 alone and K=2 when using both CyclinG2 and Sharp1, xⁱₖ the expression level of CyclinG2 or Sharp1 in the unknown sample i, \hat{μ}^{k} and \hat{σ}^{k} respectively the estimated mean and standard deviation values of the CyclinG2 and/or Sharp1 expression levels in a population with known clinical history, and wherein a signature score lower than zero indicates an increased risk of breast cancer recurrence.

The detection may be carried out by molecular and/or immunological means, where by molecular means are meant assays based on nucleic acids such as PCR, microarray analysis or Northern-blot.

The method further comprises statistical analysis of the signal through the following steps:
quality control of the acquired signal,
signal normalization,
optional rescaling of the acquired signal,
   and is preferably carried out by a software run on a computer.

The invention further provides for a kit to evaluate CyclinG2 expression alone or in combination with Sharp1 and determine the risk of cancer recurrence in a sample from a breast cancer patient, said kit preferably comprising:
a CyclinG2-specific reagent, preferably an oligonucleotide consisting in a oligonucleotide comprising at least a 13-mer oligonucleotide derived from SEQIDNO:1 or its complementary sequence;
a Sharp1-specific reagent, preferably an oligonucleotide consisting in an oligonucleotide comprising at least a 13-mer oligonucleotide derived from SEQIDNO:2 or its complementary sequence;
instructions for calculating the signature score of the unknown sample and classifying the unknown sample in the minimal signature Low group when its signature score is negative or in the minimal signature High when its signature score is positive, according to calculation defined for the method is above,
wherein classification into the minimal signature Low group is an indication of an high risk of cancer recurrence for a breast cancer patient.

According to a preferred embodiment said instructions are carried out by software. Optionally the kit may further comprise as reference standards, CyclinG2 and Sharp1 standard expression controls High and Low, as expression values or as nucleic acid samples. Said expression values or nucleic acid samples are preferably derived respectively from a non metastatic breast cancer cell line and/or from a highly metastatic cell line.

### BRIEF DESCRIPTION OF THE DRAWINGS

FIG. 1. Mutant-p53 expression promotes TGFβ pro-migratory responses.

(A) Western blot of H1299 cell lysates: parental, i.e., lacking p53 expression (null), or mutant-p53 (p53 R175H). The TGFβ signaling cascade is similarly active in both cell lines, as monitored by Smad3 phosphorylation (P-Smad3). Lamin-B is a loading control.

(B) Effect of TGFβ (5 ng/ml of TGFβ for 24 hrs) on the morphology of H1299 cells.

(C) Wound healing assays of H1299 cells showing the effects of mutant-p53 on TGFβ driven migration. Pictures were taken 30 hours after scratching the cultures.

(E) H1299 cells were seeded on transwell membranes. When indicated, cells were treated with TGFβ (4 ng/ml). The graph show the number of cells migrated through the transwell after 16 hrs. Only H1299 reconstituted with p53R175H cells acquire the ability to migrate in response to TGFβ.

FIG. 2. Mutant-p53 is required for TGFβ-driven invasion and metastasis in breast cancer mda-mb-231 cells.

(A) Western blot showing p53 protein depletion in MDA-MB-231 expressing a shRNA targeting p53 (MDA-shp53). MDA shGFP is the control cell line.

(B) Transwell assay for TGF6 dependent migration of MDA-MB-231 cell lines. This response depends on canonical Smad signaling, as attested by blockade of migration ensuing Smad4 depletion. Endogenous mutant-p53 expressed in these cells from its natural locus is required for this effect.

(C) Assay for invasive activity of MDA-MB-231 cells embedded in a drop of matrigel. Panels show pictures of the same field at different time points. Dotted is lines highlight the edges of the drop. Only control cells are able to evade from the Matrigel® (arrows). This process is dependent on TGFβ signaling as it is blocked by treatment with the TGFβR1 inhibitor SB431542 (5 μM). MDA shp53 cells are impaired in matrix degradation and evasion.

(D) MDA-MB-231 cells display spindle shape in 3D culture conditions, once embedded in Matrigel® (top panel). Arrowheads indicate lamellipodia protrusions. Conversely, MDA shp53 formed clusters of adherent, cobble-stone shaped cells (bottom panel). Inhibition of TGFβ signaling parallels the phenotypic effects of mutant-p53 depletion (data not shown).

(E and F) SCID mice were injected in the fat pad with MDA shGFP or MDA shp53 cells. (E) The rate of primary tumor growth was similar between the two cell populations. (F) Number of mice scored positive for lymphonodal metastasis.

(G, H and I) Lung colonization assays after tail vein injection of MDA-MB-231 cell lines (n of mice for each cell line=10, 1×10⁶ cells/mouse). Panels show representative immunohistochemistry for human cytokeratin in sections of lungs from mice injected with MDA shGFP (G) or MDA shp53 (H). (I) The graph quantifies the invasion of the lung parenchyma by control (shGFP) and two independent MDA shp53 clonal cell lines.

FIG. 3. Identification of a new class of candidate metastasis suppressors downstream of TGFβ/mutant-p53 in metastatic breast cancer cells

(A) Overview of TGFβ target genes from microarray analysis of MDA-MB-231 cells. The graph shows functional classification for genes regulated by TGFβ in both MDA shGFP and MDA shp53 cell lines. Many genes codes for protein involved in cell invasion, migration and metastasis (“invasive program”).

(B) Genes co-regulated by TGFβ and mutant-p53 in MDA-MB-231 cells. The table displays TGFβ induction levels for the indicated genes from microarray expression data. Differences in fold induction between MDA shGFP and MDA shp53 samples are statistically significant as indicated by q-values.

(C) Northern blot validation of ADAMTS9, Sharp1, CyclinG2, Follistatin and GPR87 as mutant-p53 dependent target of TGFβ in MDA-MB-231. When indicated (+), cells were treated for two hours with TGFβ1. GAPDH is a loading control.

(D) Regulation of Sharp1 and CyclinG2 expression by TGFβ and mutant-p53 in MDA-MB-231 cells. Northern blot analysis of MDA shGFP and MDA shp53 cells untreated or treated for two hours with TGFβ1. GAPDH is a loading control. Both genes are downregulated by TGFβ in control cells but not after mutant-p53 knockdown.

(E) Sharp1 and CyclinG2 are key effectors of the TGFβ/mutant-p53 in regulating migration. Transwell migration assay of MDA-MB-231 cells transiently transfected with the indicated siRNAs. The impairment of TGFβ-driven migration in mutant-p53 depleted cells can be rescued by concomitant depletion of Sharp1 or CyclinG2. β-Actin is a loading control.

FIG. 4. Clinical validation of the Minimal Signature as a powerful predictor of recurrence for breast cancer.

Validation of the predictive power of the minimal signature (Sharp1+CyclinG2) on a panel of five independent datasets summing-up more than 940 tumors (see Table 3 for a complete description of these data). The NKI dataset (see FIG. 6) has been analyzed separately. The analysis separates tumor samples in two groups, with coherent low or high expression of both genes, as visualized by box-plot graphs. ‘Low’ (blue) and ‘High’ (red) are the names of the minimal signature Low and minimal signature High groups, respectively.

Kaplan-Meier graphs on the left show the probability that patients, stratified according to the minimal signature, would remain free of metastases, free of recurrence, or free of disease in the analyzed breast cancer datasets. The p-value of the log-rank test reflects a significant association between minimal signature High and longer survival. Similar results were obtained using unsupervised clustering methods to generate the minimal signature Low and minimal signature High groups (data not shown).

On the right, for comparison, Kaplan-Meier survival graphs from the same tumor data stratified according to the 70 genes signature (van't Veer et al., 2002).

FIG. 5. The Minimal Signature is associated to risk of distant metastasis to both bone and lung.

Kaplan-Meier curves show the probability to remain free of lung (left) and bone (right) metastasis for MSK samples (Minn et al., 2005) stratified according to the minimal signature. The minimal signature has a statistically significant predictive is power for both organ-specific metastasis events.

FIG. 6. Analysis of CyclinG2 expression is sufficient to predict metastasis-free survival in the NKI dataset.

Expression data for the sole CyclinG2 can be used to classify tumors according to their metastatic proclivity in the NKI dataset (295 samples). As Sharp1 expression data are not available for the NKI dataset, we set a threshold value for the CyclinG2 expression on the basis of the proportion of the good prognosis patients (see Experimental Procedures for details). Box plot for CyclinG2 and Kaplan-Meier metastasis-free survival curves are obtained using this threshold value.

FIG. 7. The Minimal Signature resolves grade 2 tumors in two groups with different outcomes.

Kaplan-Meier curves showing the probability of remaining free of recurrence, disease or metastasis for patients from the Stockholm, Uppsala and NKI datasets stratified according the Nottingham histological scale (grade 1 dotted line; grade 2, violet line; and grade 3, dashed line). Grade 2 tumors (solid line) were further split in two groups by applying the minimal signature (red line: grade 2 and minimal signature High; blue line: grade2 and minimal signature Low). Notably, the High and Low groups displayed a recurrence-free survival rate similar to the grade 1 or grade 3 patients, respectively.

### DETAILED DESCRIPTION OF THE INVENTION

Definitions and Abbreviations

CyclinG2, also called CCNG2 is identified by the gene ID=901 (SEQIDNO:1). Sharp1, also called DEC2, BHLHB3, BHLHE41 (basic helix-loop-helix domain containing) is identified by the gene ID=79365 (SEQIDNO:2).

Template

Minimal signature template is obtained by measuring the expression levels of CyclinG2 alone or preferably in combination with Sharp1 in a population of tumor samples from patients with known clinical history.

A template is calculated for each different assay used to determine CyclinG2 and Sharp1 expression measure.

When both gene expression levels are measured, the template is represented by \hat{μ}^{Sharp-1}, \hat{μ}^{CyclinG2}, \hat{σ}^{Sharp-1} and \hat{σ}^{CyclinG2}, means and standard deviations of Cyclin2 and preferably Sharp1 expression levels in the population or dataset.

The expression levels of CyclinG2 and Sharp1 in two cell lines, BT20 (ATCC #HTB-19) and MDA-MB-436 (ATCC #HTB-130), representative for non-invasive and metastatic breast cancers, or other representative high and low standard expression controls, are preferably added to the population values of the template.

Standard Expression Controls

By standard expression controls are meant expression values of CyclinG2 alone or in combination with Sharp1 in non-invasive and metastatic breast cancers samples or cell lines, such as BT20 (ATCC #HTB-19) and MDA-MB-436 (ATCC #HTB-130), or other representative high and low CyclinG2 alone or in combination with Sharp1 expression standards.

Signature Score (or Expression Score)

The signature score quantifies the differences between the CyclinG2 and preferably also Sharp1 expression values in the unknown samples as compared to the template.

The signature score is defined, generally, as follows:

$${\sum }_{k = 1}^{K} \frac{{x}_{i}^{k} - {\hat{μ}}^{k}}{{\hat{σ}}^{k}}$$

being K=1 when using CyclinG2 alone and K=2 when using both CyclinG2 and Sharp-1, xⁱₖ the expression level of CyclinG2 or Sharp-1 in the unknown sample i, \hat{μ}^{k} and \hat{σ}^{k} respectively, the estimated mean and standard deviation values of the CyclinG2 and/or Sharp1 expression levels in a population with known clinical history.

For CyclinG2 and Sharp1 expression measured in combination:

$$\frac{{x}_{i}^{Sharp - 1} - {\hat{μ}}^{Sharp - 1}}{{\hat{σ}}^{Sharp - 1}} + \frac{{x}_{i}^{Cyclin G 2} - {\hat{μ}}^{CyclinG 2}}{{\hat{σ}}^{Cyclin G 2}} = Signature score for Cyclin G 2 and Sharp 1 in combination ,$$

where xᵢ^{Sharp-1}, xᵢ^{CyclinG2} are the expression levels of Sharp1 and CyclinG2 in the unknown sample i and \hat{μ}^{Sharp-1}, \hat{μ}^{CyclinG2}, \hat{σ}^{Sharp-1} and \hat{σ}^{Cyclin-G2} define the template.

When the minimal signature template is obtained by measuring the expression levels of CyclinG2 alone, the signature score is calculated as follows:

$$\frac{{x}_{i}^{Cyclin G 2} - {\hat{μ}}^{Cyclin G}}{{\hat{σ}}^{Cyclin G 2}}$$

where xᵢ^{CyclinG2} is the expression levels of CyclinG2 in the unknown sample i and \hat{μ}^{CyclinG2} and \hat{σ}^{CyclinG2} define the template.

Minimal Signature

Minimal signature High is defined a signature (expression) score higher than zero.

Minimal signature Low is defined a signature (expression) score lower than zero.

Recurrence

Recurrence is defined as the development a breast cancer related metastasis (more commonly to lung or bones) or breast cancer relapse within a period of 12 years from primary tumor surgery.

Controls

Assay controls: “assay controls” as known by the skilled man, evaluate the reliability of signal measure and acquisition by which the assay can be trusted to provide consistent results. For example, a positive “assay control” for PCR, is a known mix of nucleic acids where the PCR with the primers used, is expected to give the amplification of a DNA fragment of expected length.

Internal expression controls: the term is used, generally, to indicate housekeeping gene expression controls.

### DETAILED DESCRIPTION

The present invention is based on the experimental evidence that mutant alleles of p53 cooperates with TGFβ, sustaining its pro-invasive and malignancy responses. Indeed, mutant-p53 expression is required for invasion in vitro and for metastatic spread in vivo, highlighting a previously uncharacterized connection between these two pathways in breast cancer progression.

The pro-invasive pathway activated by TGFβ in a mutant p53 manner, involves the down-regulation of the CyclinG2 and Sharp1 genes whose lower expression levels correlates with a pro-invasive behavior of breast cancer and thus with a higher risk of cancer recurrence.

This invention shows that CyclinG2 alone or CyclinG2 together with Sharp1, henceforth Minimal Signature (MS), have predictive power comparable to more complex gene set predictors. Due to the small number of genes involved in this evaluation, the present invention can be carried out by commonly used techniques and simple PCR apparatuses.

The correlation between the minimal signature and the breast cancer recurrence or metastatic spread, has been validated through statistical analysis on several breast cancer datasets using the expression levels of these two genes; in one database, however, statistical analyses have shown that CyclinG2 alone is predictive of cancer recurrence.

The method is based on the generation of a minimal signature template using the expression levels of CyclinG2 (Gene ID=901) preferably in combination with the expression levels of Sharp1 (Gene ID=79365) from a plurality of preferably at least 50-100 of tumor patients with known clinical follow-up or available breast cancer patients datasets.

The invention discloses a method to evaluate a breast cancer patient's risk of recurrence comprising detecting the level of CyclinG2 (Gene ID=901) gene expression alone or in combination with Sharp1 (Gene ID=79365) in an unknown sample.

It preferably comprises the following steps method for evaluating the risk of “cancer recurrence” for a breast cancer patient:
(a) detecting the CyclinG2 (Gene ID=901), preferably in combination with Sharp1 (Gene ID=79365) gene expression level(s) in a sample from a breast cancer patient (i.e. measuring and acquiring a signal related to the marker genes expression);
(b) calculating a signature score for CyclinG2 alone or for, preferably, both CyclinG2 and Sharp-1 in the unknown sample, wherein said signature score is defined as:

$${\sum }_{k = 1}^{K} \frac{{x}_{i}^{k} - {\hat{μ}}^{k}}{{\hat{σ}}^{k}}$$

being K=1 when using CyclinG2 alone and K=2 when using both CyclinG2 and Sharp-1, xᵢ^{k} the expression level of CyclinG2 or Sharp-1 in the unknown sample i, \hat{μ}^{k} and \hat{σ}^{k} respectively the estimated mean and standard deviation values of the CyclinG2 and or Sharp-1 expression levels in a population with known clinical history,
(c) classifying the unknown sample in a minimal signature Low group when said signature score is lower than 0 or to a minimal signature High group when said signature score is higher than 0, wherein the assignment to the Low group correlates with a high risk of recurrence.

The sample may be a breast cancer biopsy or a lymph node and either the tissue section or the nucleic acids, preferably the mRNA or cDNA isolated from such a sample.

The high predictive power of the method of the present invention, measuring CyclinG2 (Gene ID=901) alone, or preferably in combination with Sharp1, is particularly surprising because this is a signature of only two genes over more than 400 regulated by TGFβ and none of the already proposed signatures comprises any one of the two genes according to the present invention, whose prognostic use for breast cancer recurrence is described here for the first time.

The minimal signature template is prepared by collecting gene expression data (i.e. CyclinG2 and, preferably also Sharp1) from a population of patients whose clinical data and survival times at 5-12 years are known.

The detection of one or preferably the two markers genes in the unknown sample, is preferably carried out, at the same time and with the same reagents, in a control for the High expression level standard of each of the genes (control High CyclinG2 and control High Sharp1) and in a control for the Low expression (control Low CyclinG2 and control Low Sharp1).

Standard expression controls High and Low may be either derived from known patients or from cell lines that are representative for non-invasive or metastatic breast cancers (e.g., BT20 or MDA-MB-436) respectively. BT20 (ATCC #HTB-19) and MDA-MB-436 (ATCC #HTB-130) are two different breast cancer cell lines representative for non-invasive and metastatic breast cancers, respectively. BT20 expresses high levels of both genes, and, conversely, in MDA-MB-436 Sharp1 and CyclinG2 are down-regulated. Thus these two cell lines may provide easy-to-obtain High (BT20) and Low (MDA-MB-436) standard expression controls for the proposed method.

In addition, at least one internal expression control for normalization purposes, is measured in the same reaction.

The selection of the internal expression control depends on the experimental technique used for monitoring the expression levels; normalization of the expression data may be based on computational methods (as scaling to average expression levels of all genes or quantile normalization) when using microarrays or on the expression levels of internal controls for molecular techniques based on nucleic acid, i.e. PCR or Northern-blot. Housekeeping genes commonly used to this purposes, for example in PCR, are selected among GAPDH, β-actin etc., which are constitutively expressed. For immunodetection based methods, internal controls will be preferably selected among LaminB or GAPDH immunoreactivity.

Moreover, further assay controls as known by the skilled man, are preferably included in the method to evaluate the reliability of steps a) and b) providing a control through which the assay can be trusted to provide consistent results.

For example a positive assay control for PCR, is a known mix of nucleic acids where the PCR with the primers used, is expected to give the amplification of a DNA fragment of expected length.

Measurement of the CyclinG2 and/or the Sharp1 gene expression levels are assessed by any known state-of-the-art method, for example by molecular means based on molecular selection (i.e. selective amplification or hybridization) and/or by immunological means.

Molecular selection (i.e. selection by sequence specific hybridization with sequence specific probes or primers for CyclinG2 and/or Sharp1) is usually followed by a separation step of the polynucleotide molecules targeted and/or amplified, on the basis of the molecular weight, followed by quantification, for example by densitometry or by visual inspection, then by data normalization with any state-of-the-art computational method for example by linear scaling or non-linear normalization, and, preferably, by comparison with standard expression controls.

Preferably, comparison of the sample values with the minimal signature template is carried out by calculating the signature score.

More in general however, the invention is based on the definition that, when the is expression levels of CyclinG2, alone or preferably in combination with Sharp1 gene in a sample, define a signature score which is lower than zero, this represents an indication that there is an increased risk of (breast) cancer recurrence.

Statistical analysis to compare and/or differentiate an individual having one phenotype (for example an unknown sample) from other individuals having a second phenotype (for example the minimal signature template) is preferably used. Preferably this is carried out by a software.

Thus, according to a preferred embodiment, the method of the invention comprises a step b) carried out by a software running on a computer, which retrieves the stored template, quantifies the signature score of the sample through the marker(s) expression level signal(s) and assigns the unknown sample to High or Low minimal signature groups (as defined in step b) above).

More preferably, the analysis of the signals (expression data) which have been acquired (according to step a) above) is carried out through the following additional steps:
data quality control, on the basis of the assay control,
data normalization according and depending to the technology used to quantify gene expression levels,
preferably, data rescaling on the basis of the standard expression controls, for example by linear or non-linear scaling.

After the signal has been suitably analysed, the template is retrieved, the signature score of the sample is calculated and the unknown sample is assigned to minimal signature High or Low groups (as defined in step c)) above.

When the signature template is stored on a computer, or on computer readable media, and the software is used in prognosis-correlated signatures, the signature template is compared to the signature score from the sample. This means that in other words, the expression levels of one or both the 2 marker genes in the sample, suitably and preferably analysed, are compared to the distribution of the expression levels of the same genes in the minimal signature, as determined from a pool of samples from patients with known prognosis (i.e., a pool of numerically suitable samples usually comprised from at least 50 to 100) comprising samples is from patients or, alternatively or in addition, from cell lines that are representative for non-invasive and metastatic breast cancers.

Then, the unknown sample is classified as having a good prognosis for cancer recurrence if the levels of expression of one or both the 2 marker genes determine a signature score higher than zero. Conversely, unknown sample whose signature score is lower than zero are classified by the software as from patients having a poor prognosis.

Although the method is preferably carried out by a software, the method is not limited to this embodiment: in fact the assignment to the High and Low expression group may be also carried out by visual inspection of the sample absolute expression signal, in the presence of the controls known by the skilled man, and by visually or numerically comparing this to the High and Low signature template (or standard expression controls as defined above).

Preferably, to increase the sensitivity of the comparison, the signal related to the expression levels, may be normalized e.g. by using different techniques, such as the average expression level of a set of control genes.

In different embodiments, markers expression level are normalized by the mean or median level of expression of a set of control markers (internal expression controls are, for nucleic acid based assays: GAPDH or β-Actin; for immunologically based assays: GAPDH and LaminB).

In another specific embodiment, the normalization is accomplished by standardization of the marker levels. The expression level data may be transformed in any convenient way, but, preferably, the expression signals are log transformed before normalization and comparison are carried out. Normalized values are then compared to the minimal signature template, which is composed of the normalized and/or transformed expression levels of the same marker genes, collected using the same experimental technique and protocols from a suitable lo pool of tumor patients with known clinical follow-up and from different breast cancer cell lines representative for non-invasive and metastatic breast cancers (e.g., BT20 and MDA-MB-436, respectively).

As an example, if the markers are represented by probes on a microarray, the expression level of each of the markers may be normalized by the mean or median expression level across all of the genes represented on the microarray, including any non-marker (i.e. non CyclinG2 and non Sharp1) genes.

As said above, measurements of the expression levels can be carried out by any known method: molecular means comprises for example PCR (standard or Real-Time), Northern blot or microarray analysis.

By Northern blot, total RNA samples are separated by electrophoresis according to the size and hybridization is carried out with labeled probes specific for the CyclinG2 and/or Sharp1.

PCR, or RT-PCR comprises as a preliminary step, the reverse transcription of a RNA sample in cDNA, can be carried out by using PCR primers identified from the published sequence of the CyclinG2 and Sharp1 by standard sequence analysis with known and available software, for example by Primer3 (http://primer3.sourceforge.net).

Preferred CyclinG2 and Sharp1 forward and reverse primers for the PCR-based molecular method of the invention are shown in the following table comprising PCR primers also for amplification of preferred internal control genes:

Standard PCR primers

| Name | Sequence |
|---|---|
| Actin for | ATGAAGTGTGACGTTGACATCCG |
| Actin rev | GCTTGCTGATCCACATCTGCTG |
| p53 for | CTGGCCCCTGTCATCTTCTGTC |
| p53 rev | CACGCAAATTTCCTTCCACTCG |
| SHARP1 for | GCATGAAACGAGACGACACC |
| SHARP1 rev | CGCTCCCCATTCTGTAAAGC |
| CyclinG2 for | CCTCCCAGTGATCAAGAGTGC |
| CyclinG2 rev | TCCCTCCTCCCCAAAGTAGC |

For quantitative PCR (Q-PCR) the following preferred primers are used:

Q-PCR primers

| Name | Sequence |
|---|---|
| GAPDH for | AGCCACATCGCTCAGACAC |
| GAPDH rev | GCCCAATACGACCAAATCC |
| SHARP1 for | CGTCTTTGGAGTTGACATGG |
| SHARP1 rev | GGGCAGCTTTGAGAACTAGC |
| CyclinG2 for | TGGACAGGTTCTTGGCTCTT |
| CyclinG2 rev | GATGGAATATTGCAGTCTTCTTCA |

One of the most widely used ways of gene expression analysis is by (micro)array. As for any other kind of expression data measurement, the statistical analysis of the unknown sample comprises the preliminary evaluation of the minimal signature template for the CyclinG2 (Gene ID=901) alone or preferably in combination with the Sharp1 (Gene ID=79365), by collecting a suitable number (at least 50-100) of measurements from breast cancer patients with known clinical follow-up.

These data, i.e. the minimal signature template, as said above, may be defined in advance and the relevant information stored on a computer for the next sample analysis.

The method of the invention has been validated in the following breast cancer microarray datasets:

| | Microarray | | | |
|---|---|---|---|---|
| Study | platform | Samples | Data source | Reference |
| Stockholm | Affymetrix | 156 | GEO GSE1456 | (Pawitan et al., |
| | HG-U133A | | | 2005) |
| NCI | Affymetrix | 187 | GEO GSE2990 | (Sotiriou et al., |
| | HG-U133A | | | 2006) |
| EMC | Affymetrix | 286 | GEO GSE2034 | (Wang et al., |
| | HG-U133A | | | 1998) |
| Uppsala | Affymetrix | 236 | GEO GSE3494 | (Miller et al., |
| | HG-U133A | | | 2005) |
| MSK | Affymetrix | 82 | GEO GSE2603 | (Minn et al., |
| | HG-U133 | | | 2005) |
| NKI | Agilent, | 295 | http://www.rii.com/ | (van ‘t Veer et |
| | Rosetta | | publications/2002/ | al., 2002; van |
| | Inpharmatics | | nejm.html; | de Vijver et al., |
| | | | http://microarray- | 2002; Fan et |
| | | | pubs.stanford.edu/ | al., 2006) |
| | | | wound_NKI/ | |
| | | | explore.html | |

Classification within one of the two groups of values with either high or low simultaneous expression scores of Sharp1 and CyclinG2, is preferably carried out by summarizing the standardized expression levels of Sharp1 and CyclinG2 into a combined score with zero mean.

Tumors are classified as minimal signature Low if the combined score is negative and as minimal signature High if the combined score is positive:

$$minimal signature Low -> \frac{{x}_{i}^{Sharp - 1} - {\hat{μ}}^{Sharp - 1}}{{\hat{σ}}^{Sharp - 1}} + \frac{{x}_{i}^{Cyclin G 2} - {\hat{μ}}^{CyclinG 2}}{{\hat{σ}}^{Cyclin G 2}} \leq 0$$

$$minimal signature High -> \frac{{x}_{i}^{Sharp - 1} - {\hat{μ}}^{Sharp - 1}}{{\hat{σ}}^{Sharp - 1}} + \frac{{x}_{i}^{Cyclin G 2} - {\hat{μ}}^{Cyclin G 2}}{{\hat{σ}}^{Cyclin G 2}} > 0$$

where xᵢ^{Sharp-1}, xᵢ^{CyclinG2} are the expression levels of Sharp1 and CyclinG2 in sample i and \hat{μ}_{Sharp-1}, \hat{μ}^{CyclinG2}, \hat{σ}^{Sharp-1} and \hat{σ}^{CyclinG2} are the estimated means and standard deviations of Sharp1 and CyclinG2 calculated over an entire dataset and represent the minimal signature template

In the case of the NKI dataset, samples had to be classified in High and Low is groups based on CyclinG2 data only, which represents thus the minimal requirement for the prognostic validity of the method. In this dataset (295 tumors), the stratification based on the sole CyclinG2 remains predictive of metastasis.

In fact, when the expression levels of CyclinG2 alone are used to define the minimal signature template, tumors are classified as minimal signature Low if the CyclinG2 score is negative and as minimal signature High if the CyclinG2 score is positive according to the following calculation:

$$minimal signature Low -> \frac{{x}_{i}^{CyclinG 2} - {\hat{μ}}^{Cyclin G 2}}{{\hat{σ}}^{CyclinG 2}} \leq 0$$

$$minimal signature High -> \frac{{x}_{i}^{Cyclin G 2} - {\hat{μ}}^{CyclinG 2}}{{\hat{σ}}^{CyclinG 2}} > 0$$

where xᵢ^{CyclinG2} is the expression levels of CyclinG2 in the unknown sample i and \hat{μ}^{CyclinG2} and \hat{σ}^{CyclinG2} define the template.

The risk of cancer recurrence is accordingly evaluated as “high” for the minimal signature Low expression group.

The same analysis briefly described above and better detailed in the experimental part for validating the two markers, can be carried out for any new or different dataset; therefore according to a further embodiment, the present invention relates to a method for analyzing a breast cancer microarray dataset with the expression values of CyclinG2 alone or in combination with Sharp1.

By applying the method above to all the above mentioned datasets, the prognostic method of the invention has been demonstrated, strikingly, to be highly predictive for breast cancer recurrence in the group expressing low levels of the minimal signature which displays a significant higher probability to develop recurrence when compared to the “High” group (p-values ranged from 0.02 to 3E-05, depending on the datasets) when tested using the univariate Kaplan-Meier survival analysis.

Interestingly, the Minimal Signature based on both CyclinG2 and Sharp1 expression levels performed comparably to the 70-genes profile described in van't Veer et al., 2002 in stratifying patients according to their clinical outcome.

The advantages of using a minimal signature based on only two genes instead of 70 genes are clearly evident.

A further advantage of the method of the present invention is that the expression of CyclinG2 and Sharp1 are statistically correlated to the risk of distant metastasis to both bone and lung, and thus are independent from the site of secondary tumor formation.

Moreover, although the simplest way the method can be carried out, is by PCR, for which it is required only a minimal apparatus, such as a PCR termocycler and a tank for DNA separation by gel electrophoresis, the invention is not limited to this embodiment, but relates to all the available methodologies commonly used to measure gene expression levels, when applied to the detection of CyclinG2 expression levels alone or in combination with Sharp1, as prognostic markers for the risk of breast-cancer recurrence.

Therefore, the method of the present invention can be based on any one of the following techniques for gene expression analysis, such as:
standard PCR technique,
Real time PCR (or Q-PCR, with Taq man or Sybr Green technology),
microarray, possibly in combination with sequences specific for other genes,
deep sequencing (t Hoen et al., 2008), possibly in combination with sequences specific for other genes,
northern blot,
immunohistochemistry with available antibodies against CyclinG2 and/or Sharp1,
immunoblot,
   to measure the gene expression levels on specific mRNA, or on the protein product.

According to the preferred technique for expression level measurements, Quantitative PCR or Reverse Transcribed mRNA PCR, the CyclinG2 detecting reagent is a CyclinG2-specific oligonucleotide, consisting in an oligonucleotide comprising at least a 13-mer oligonucleotide derived from SEQIDNO:1 or its complementary sequence.

For immunodetection, preferably, an anti-CyclinG2 alone or in combination with Sharp1 specific antibodies are used.

Therefore summarizing, according to the preferred embodiment of the method which comprises also the detection of Sharp1 expression levels, the specific detecting reagent is selected from the group consisting of: a Sharp1 specific oligonucleotide, consisting in an oligonucleotide comprising at least a 13-mer oligonucleotide derived from SEQIDNO:2 or its complementary sequence, or an anti-Sharp1 specific antibody.

A further embodiment of the invention is a kit for evaluating a breast cancer patient's risk of cancer recurrence, comprising CyclinG2 and preferably also Sharp1 gene expression specific detection means, i.e. CyclinG2-specific oligonucleotides or probes, consisting in poly- or oligonucleotide comprising at least a 13-mer oligonucleotide derived from SEQIDNO:1 or its complementary sequence, and preferably Sharp1-specific oligonucleotide, consisting in poly- or oligonucleotide comprising at least a 13-mer oligonucleotide derived from SEQIDNO:2 or its complementary sequence.

As a further embodiment the invention is related to a kit for evaluating the expression of CyclinG2 alone or in combination with Sharp1 in a sample from a breast cancer patient comprising at least a CyclinG2-specific reagent, preferably is an oligonucleotide comprising at least a 13-mer derived from SEQIDNO:1 or its complementary sequence; preferably also a Sharp1-specific reagent, preferably an oligonucleotide comprising at least a 13-mer derived from SEQIDNO:2 or its complementary sequence; instructions for analysing an unknown sample specifying the criteria for assignment of the unknown sample measurement to a minimal signature High or Low group as defined above. According to a preferred embodiment, a software for the statistical analysis and comparison of the expression data (the sample signature score) to the minimal signature template as defined above, wherein assignment to the minimal signature Low group correlates with an increased risk of cancer recurrence in a breast cancer patient.

The kit may further comprise as standard expression controls, CyclinG2 and Sharp1 expression controls High and Low, i.e. CyclinG2 and Sharp1 expression values measured in the cell lines BT20 and MDA-MB-436, respectively and dilution or assay buffers.

Specific reagents, useful for each of the gene expression detection methods used, may be commercially available reagents, or custom made, provided that they are specific for CyclinG2 and/or Sharp1.

Antibodies, either preferably purified polyclonal or monoclonal, or oligonucleotides may be preferably labeled with fluorochromes, chemiluminescent labels or chromogens; polynucleotides, can be used in Northern Blot after having been labeled, for example with ³²P.

Specific antibodies may be directly labeled or detected by using a secondary labeled antibody.

The kit further comprises instructions for use reporting the criteria for assigning each sample measurement to a high or low minimal signature where low minimal signature correlates with an increased risk of breast cancer recurrence, or preferably. Preferably the above specified calculation are carried out by software.

The kit may comprise assay controls, consisting in a negative and a positive sample, or reagents to detect internal expression controls and, optionally, nucleic acid extraction reagents.

According to a preferred embodiment the PCR primer pair for CyclinG2 expression is level detection are the following: CyclinG2 (forward): 5′ CCTCCCAGTGATCAAGAGTGC 3′ CyclinG2 (reverse): 5′ TCCCTCCTCCCCAAAGTAGC 3′; for Sharp1 (forward): 5′ GCATGAAACGAGACGACACC 3′ and (reverse): 5′ TCCCTCCTCCCCAAAGTAGC 3′.

Primers performing comparatively can be identified by known technologies.

Semi-quantitative PCR (RT-PCR) is typically carried out by retrotranscribing a Poly A⁺ RNA purified from total RNA extracted from a sample using as an internal expression control the GAPDH sequence, as known in the art.

A densitometric analysis or visual inspection provides for the expression level of each gene and a comparison with standard expression controls is carried out to define a low expression group for CyclinG2 alone or in combination with Sharp1.

According to an alternative embodiment, the kit comprises means for the immunological detection of the CyclinG2 and Sharp1 expression, such as specific antibodies and relevant controls.

The results provided by the method of the invention propose a first stratification of the risk of recurrence for a breast cancer patient.

As stated above, the prognostic indication for CyclinG2 and Sharp1 represents one of the most significant index for the physician, who has however to complete the prognostic evaluation with other known prognostic and predictive factors in breast cancer, such as age, tumor size, axillary lymph node status, histological tumor type, pathological grade and hormone receptor status.

In fact, as reported in better details in the Experimental Part, Example 6, the multivariate Cox proportional-hazards analysis on a 187 tumors dataset from National Cancer Institute (Sotiriou et al., 2006) of other predictors commonly used in the clinical practice, including tumor diameter, estrogen-receptor status (ER positive vs. negative), nodal status (positive vs. negative), tumor grade (grade 2 vs. grade 1 and grade 3 vs. grade 1) and treatment status (tamoxifen vs. none) in Model 2, is highly significant (p=0.0054) for the Minimal Signature (Table 4).

The minimal signature, thus, results a significant predictor of recurrence-free survival, adding new prognostic information beyond the one provided by the standard clinical predictors. Moreover, the minimal signature adds prognostic value not only to the multivariate model but also to any model calculated using any single clinical predictor. Indeed, the difference between the residual deviance of the model obtained using a single clinical variable plus the minimal signature (e.g., nodal status+minimal signature) and the residual deviance of the model obtained using only a clinical variable, is significant for each clinical predictor.

Moreover, the method of the invention is particularly useful to gain prognostic indication for patients representing more than 50% of the breast cancer patients where by traditional prognostic markers is confidentially assigned either an obviously poor or a clearly good outcome.

A particularly relevant point of the present method is that it usefully applies to tumors classified as intermediate (grade 2) by the Nottingham scale which represent the majority of tumors and whose prognosis is uncertain (Ivshina et al., 2006). When applied to grade 2 tumors of multiple independent datasets, the minimal signature stratified grade 2 samples into two groups with outcomes comparable to grade 1 and grade 3, respectively.

The resolution achieved represents thus a preferred embodiment of the method of the invention as applied to the stratification of breast tumor patients classified as Grade 2 according to Nottingham scale for a more correct classification and possibly, assignment to different therapeutic categories or clinical trials.

Experimental Part

Material and Methods

Cell Cultures and Transfections

H1299 and the derived cell line expressing mutant p53 R175H are a gift of G. Blandino (Strano et al., J Biol Chem 2002).

H1299 non-small lung carcinoma cells were maintained in DMEM, 10% serum, 1 mM glutamine. TGFβ treatments were done in DMEM 0.2% serum (TGFβ was provided from Peprotech). p53R175H H1299 cells express stably transfected plasmids coding for ponasterone-inducible cDNAs for a mutant p53R175H allele. p53 expression was induced by incubating cells with Ponasterone-A (Alexis, 3 mM) for 16 hours before treatments.

MDA-MB-231 (ATCC #HTB-26) were maintained in a 1:1 mixture of DMEM and F12 (DMEM/F12) supplemented with 10% serum, 2 mM glutamine.

For TGFβ treatments cells were serum starved for 24 hours and then treated with TGFβ1 (5 ng/ml) in DMEM/F12 without serum.

For siRNA (si: Small interfering RNA) transfection, dsRNA oligos (10 picomoles/cm²) were transfected using the RNAi Max reagent (invitrogen). A list of the sequences targeted by siRNA and shRNAs (Sh: small hairpin RNA or short hairpin RNA) is shown in table 1.

TABLE 1
Sequences targeted by siRNAs and shRNAs

| Target Gene | Sequence (sense) |
|---|---|
| GFP | CAAGCTGACCCTGAAGTTC |
| Human p53 | GACTCCAGTGGTAATCTAC |
| p53 | CCGCGCCATGGCCATCTACA |
| Smad4 | GTACTTCATACCATGCCGA |
| Sharp1 A | GCTTTAACCGCCTTAACCG |
| Sharp1 B | CGAGACGACACCAAGGATA |
| CyclinG2 A | GAGTCGGCAGTTGCAAGCT |
| CyclinG2 B | AGAATACTCGGCTAGGCAT |
| Control | TTCTCCGAACGTGTCACGT |

Generation of Stable Cell Lines

Small-hairpin-RNA (shRNA) expression constructs were generated by cloning annealed DNA oligonucleotides in pSUPER-retro-puro (OligoEngine). All plasmids were controlled by sequencing.

For stable knock-down, retroviral particles were obtained by transfecting plasmids for expression of shRNAs (pSuperRetro) and VSV envelope in 293gp (gift from M. Tripodi) with calcium-phosphate. Two days after transfection, surnatants were collected, filtered and used to infect of MDA-MB-231. After selection for puromycin resistance, transduced cells were verified for downregulation of the target protein.

Migration and Invasion Assays

For wound-closure experiments, H1299 cells were plated in 6-well plates and cultured to confluence. Cells were scraped with a p200 tip (time 0), transferred to low serum and treated as described.

Transwell migration assay were performed in 24 well PET inserts (Falcon 8.0 mm pore size) for migration assays. For MDA-MB-231, cells were plated in 10 cm dishes, transfected with siRNA and, after 8 hours, serum starved overnight. Then, 50000 or 100000 cells were plated in transwell inserts (at least 3 replicas for each sample) and either left untreated or treated with TGFβ1 (5 ng/ml). For H1299, cells is were plated in the transwell in 10% serum but then changed to 0.2% serum. For both cell lines, cells in the upper part of the transwells were removed with a cotton swab; migrated cells were fixed in PFA 4% and stained with Crystal Violet 0.5%. Filters were photographed and the total number of cells counted. Every experiment was repeated at least 3 times independently.

For matrigel invasion assay shown in FIG. 2C, MDA-MB-231 and derivative cell lines were resuspended in drops (100 ml) of Matrigel Growth Factor Reduced (BD Biosciences), diluted 1:2 in DMEM/F12.

In Vivo Metastasis Assays

Mice were housed in Specific Pathogen Free (SPF) animal facilities and treated in conformity with approved institutional guidelines (University of Padova). For xenograft studies of breast cancer metastasis, shGFP- or shp53-MDA-MB-231 cells (1×10⁶ cells/mouse) were unilaterally injected into the mammary fat pad of SCID female mice, age-matched between 5 and 7 weeks. After six weeks, mice were sacrificed and examined for metastases to lymph nodes. Macroscopic metastases to other organs were infrequent (liver, lung, peritoneum). Tumor growth in the injected site was monitored by repeated caliper measurements. For lung colonization assays, cells were resuspended in 100 ml of PBS and inoculated in the tail vein of SCID mice. Four weeks later, animals were sacrificed and lungs removed for the subsequent histological analysis.

Histology and Immunohistochemistry

Tissues for histological examination were fixed in 4% buffered formalin, dehydrated and embedded in paraffin by standard methods.

For the experiments depicted in FIGS. 2G-I, serial sections of the lungs, cut at a distance of 150 mm from each other, were first stained with Hematoxylin and Eosin (H&E) and then processed for human cytokeratin expression with monoclonal mouse anti-human Cytokeratin, clone MNF116 (Dako). Immunohistochemical staining was performed using an indirect immunoperoxidase technique (Bond Polymer Refine Detection; Vision BioSystems, UK).

We quantified the cytokeratin-positive area in 5 serial sections per lung. The area covered by tumor cells was determined using ImageJ software (NIH), from 4 non-overlapping fields (covering 50-80% of each section) per section.

Antibodies and Western Blotting

Western blot analysis was performed as previously described (Piccolo et al., 1999). Briefly, proteins were resolved in 10% NuPage® gels (Invitrogen) and transferred to ImmobilonP® membranes (Millipore). Chemiluminescence was revealed using Supersignal West-pico® and -dura HRP substrates (Pierce). Anti-human p53 DO-1 monoclonal antibodies and anti-Lamin polyclonal antibodies were purchased from Santa Cruz biotechnology. Anti-phospho-Smad3 polyclonal antibody was from Cell Signaling.

Northern Blotting

Total RNA was extracted from cells plated in 6 cm dishes with Trizol (Invitrogen). 10 mg of total RNA per sample were loaded and separated in a 6% formaldehyde/1% agarose gel, blotted by upward capillary transfer onto GeneScreenPlus (Perkin Elmer) and UV crosslinked. Membranes were pre-hybridized 5 hrs at 42° C. with ULTRAhyb-Oligo solution (Ambion), and hybridized with ³²P-labeled DNA probes o.n. at 42° C. Membranes were washed at 68° C. with 2×SSC/0.5% SDS solutions and exposed for autoradiography. All probes were obtained by random-primer amplification. Sharp1, CyclinG2 and Follistatin probe templates were obtained from RZPD EST (HU3_p983B0120D, HU3_p983D0140D2 and RZPD EST HU3_p983D0113D2 respectively). GPR87 and ADAMTS9 probes were obtained cloning RT-PCR products. All probes were validated by sequencing.

RT-PCR

Poly(A)⁺-RNA was retrotranscribed with M-MLV Reverse Transcriptase (Invitrogen) and oligo-d(T) primers following total RNA purification with Trizol (Invitrogen). For standard RT-PCR 2 ul of each cDNA sample is aliquoted to PCR tubes and a master PCR mix for EXTaq (Finnzymes) is then added. Cycling conditions are: 94° C. 30 sec, 55° C. 30 sec, 72° C. 60 sec (Cordenonsi et al., 2003). A list of all PCR primers is shown in Table 2.

TABLE 2
RT (Reverse Transcribed) and Q
(quantitative) PCR primers

| | |
|---|---|
| Name | Sequence |

standard PCR primers

| | |
|---|---|
| Actin for | ATGAAGTGTGACGTTGACATCCG |
| Actin rev | GCTTGCTGATCCACATCTGCTG |
| p53 for | CTGGCCCCTGTCATCTTCTGTC |
| p53 rev | CACGCAAATTTCCTTCCACTCG |
| SHARP1 for | GCATGAAACGAGACGACACC |
| SHARP1 rev | CGCTCCCCATTCTGTAAAGC |
| CyclinG2 for | CCTCCCAGTGATCAAGAGTGC |
| CyclinG2 rev | TCCCTCCTCCCCAAAGTAGC |

Q-PCR primers

| | |
|---|---|
| GAPDH for | AGCCACATCGCTCAGACAC |
| GAPDH rev | GCCCAATACGACCAAATCC |
| SHARP1 for | CGTCTTTGGAGTTGACATGG |
| SHARP1 rev | GGGCAGCTTTGAGAACTAGC |
| CyclinG2 for | TGGACAGGTTCTTGGCTCTT |
| CyclinG2 rev | GATGGAATATTGCAGTCTTCTTCA |

Q-PCR for CyclinG2 and GAPDH was done by using 7500 Real-Time PCR System (Applied Biosystems) with DyNAmo HS SYBR Green (Finnzymes).

Microarray Analysis

MDA shGFP and shp53 cells were serum-starved for 24 hours, and then either left untreated or treated with TGFβ1 (5 ng/ml for 3 hours) in DMEM/F12 without serum. Four replicas were prepared for each of the four conditions (untreated shGFP, TGFβ-treated shGFP, untreated shp53, TGFβ-treated shp53) for a total of 16 samples. Total RNA was extracted using Trizol (Invitrogen) according to the manufacturer's instructions. Sample preparation for microarray hybridization was carried out as described in the Affymetrix GeneChip® Expression Analysis Technical Manual. Briefly, 15 μg of total RNA were used to generate double-stranded cDNA (Invitrogen). Synthesis of Biotin-labeled cRNA was performed using the BioArray™ HighYield™ RNA Transcript Labeling Kit (ENZO Biochem, New York, N.Y.). The length of the cRNA fragmentation was confirmed using the Agilent 2100 Bioanalyzer (Agilent Technologies). Four biological mRNA replicates for each group were hybridized on Affymetrix GeneChip® Human Genome HG-U133 Plus 2.0 arrays.

All data analyses were performed in R using Bioconductor libraries and R statistical packages (http://www.r-project.org/, R Development Core Team, 2008). Specifically, BioConductor packages affyQCReport and AffyPLM were used for standard Affymetrix quality-control procedures. Probe level signals have been converted to expression values using robust multi-array average procedure rma (Irizarry et al., 2003). In RMA, PM values have been background adjusted, normalized using quantile normalization, and expression measure calculated using median polish summarization. RMA data with a standard deviation lower than the mean standard deviation of all log signals in all arrays (e.g., 0.2) have been filtered out. The filtered data set resulted in 22644 probesets used for further analysis. Differentially expressed genes have been identified using Significance Analysis of Microarray samr (Tusher et al., 2001). SAM is a statistical technique for finding significant genes in microarrays while controlling the False Discovery Rate (FDR). SAM uses repeated permutations of the data to determine if the expression level of any genes is significantly related to the physiological state and the significance is quantified in terms of q-value (Storey, 2002), i.e. the lowest False Discovery Rate at which a gene is called differentially expressed.

Identification of TGF-β Target Genes

To identify genes whose expression is modified by TGFβ, we compared the expression profile of TGFβ treated MDA-MB-231 cells (either shGFP or shp53) with their untreated controls and selected those transcripts whose q-value was ≦0.1. This selection was further refined setting the lower limit for TGFβ fold induction (or reduction) to 1.5. Using this combined filter, we were able to identify 447 genes differentially regulated between the untreated and TGFβ treated MDA-MB-231 samples. Differentially expressed genes were functionally classified according to DAVID (http://david.abcc.ncifcrf.gov/), the Kyoto Encyclopedia of Genes and Genomes (KEGG; http://www.genome.jp/kegg/) and NCBI Gene databases (NCBI; http://www.ncbi.nlm.nih.gov/sites/entrez?db=gene). Out of 292 genes associated with known functions, 147 genes were reported to be involved in cellular movements, invasive processes and metastasis. Genes that were regulated by TGFβ1 in a mutant-p53 dependent manner were identified as those displaying a significant regulation by TGFβ in shGFP, but not in p53-depleted cells (q-value≦0.1, see FIG. 3B). The resulting 5 genes were validated by Northern blot analysis.

### EXAMPLE 1

### Effects of Mutant-p53 on the Cellular Response to TGFβ

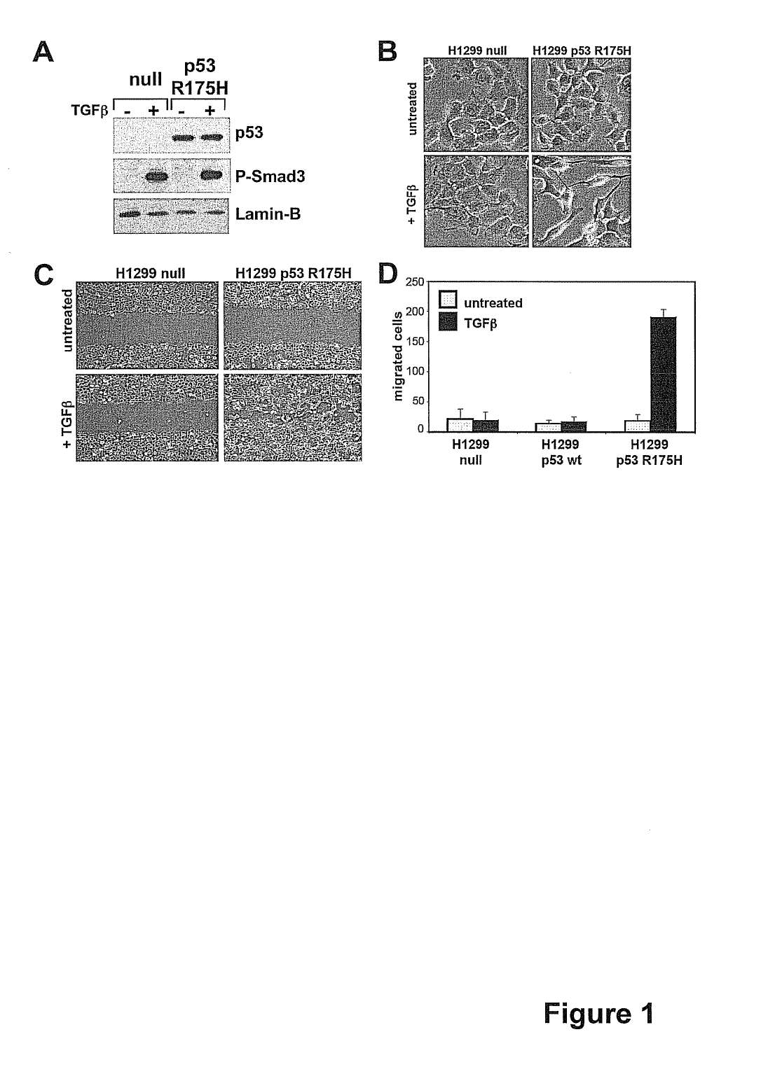

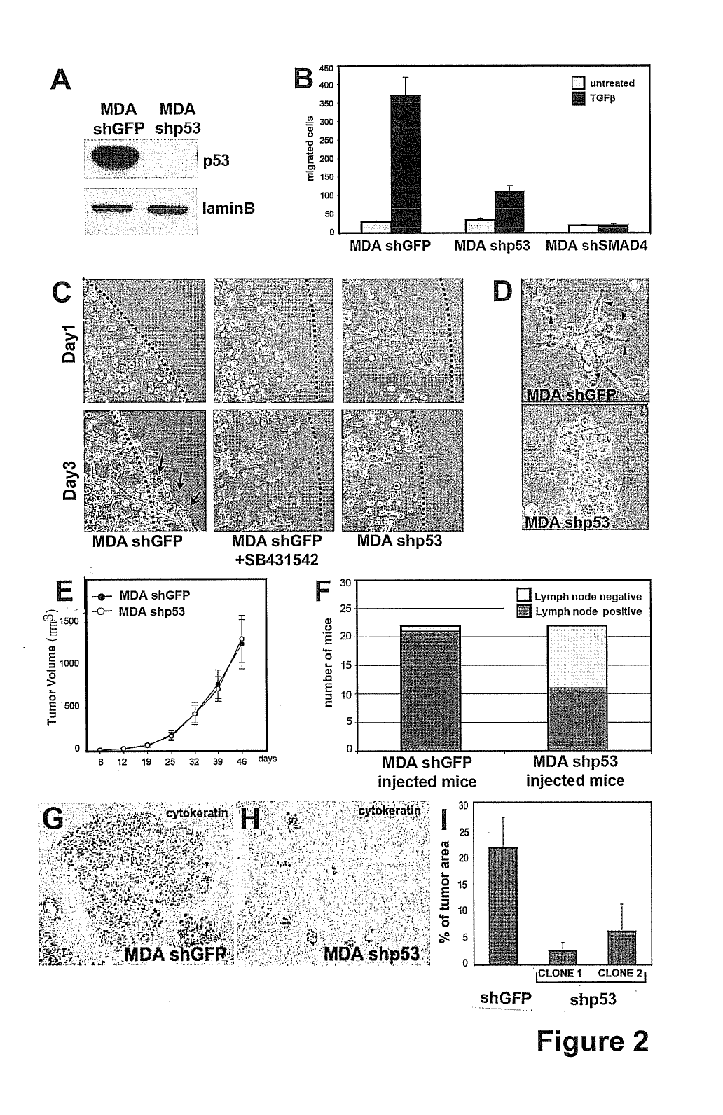

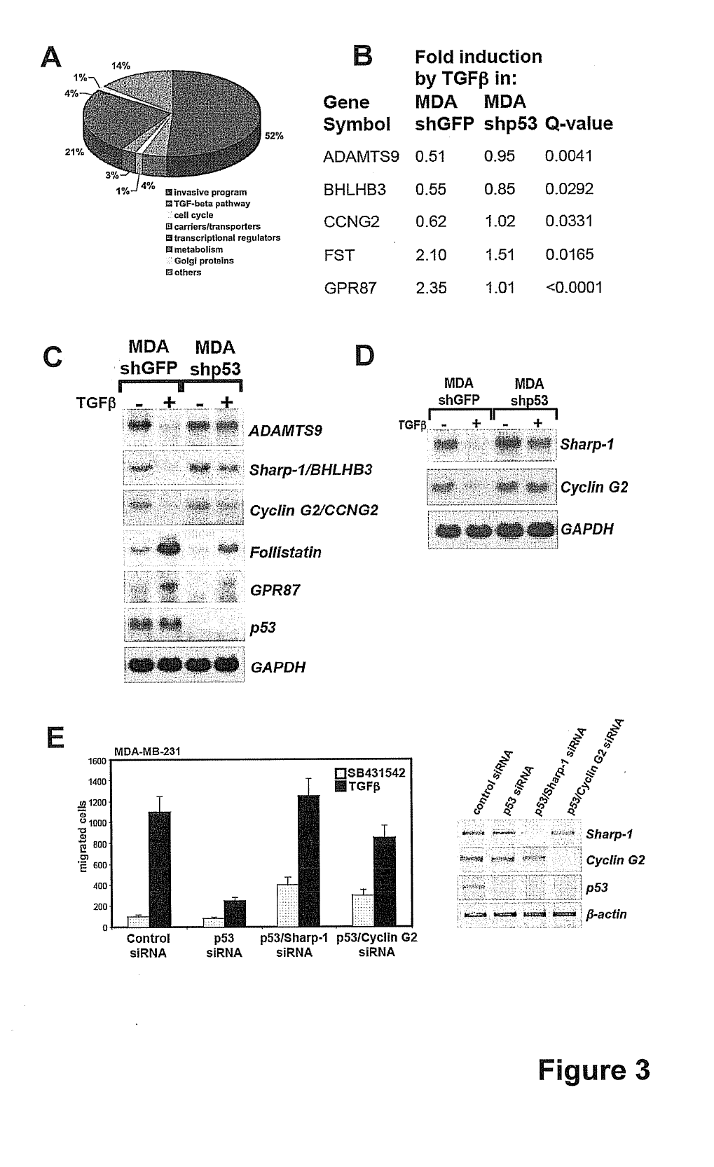

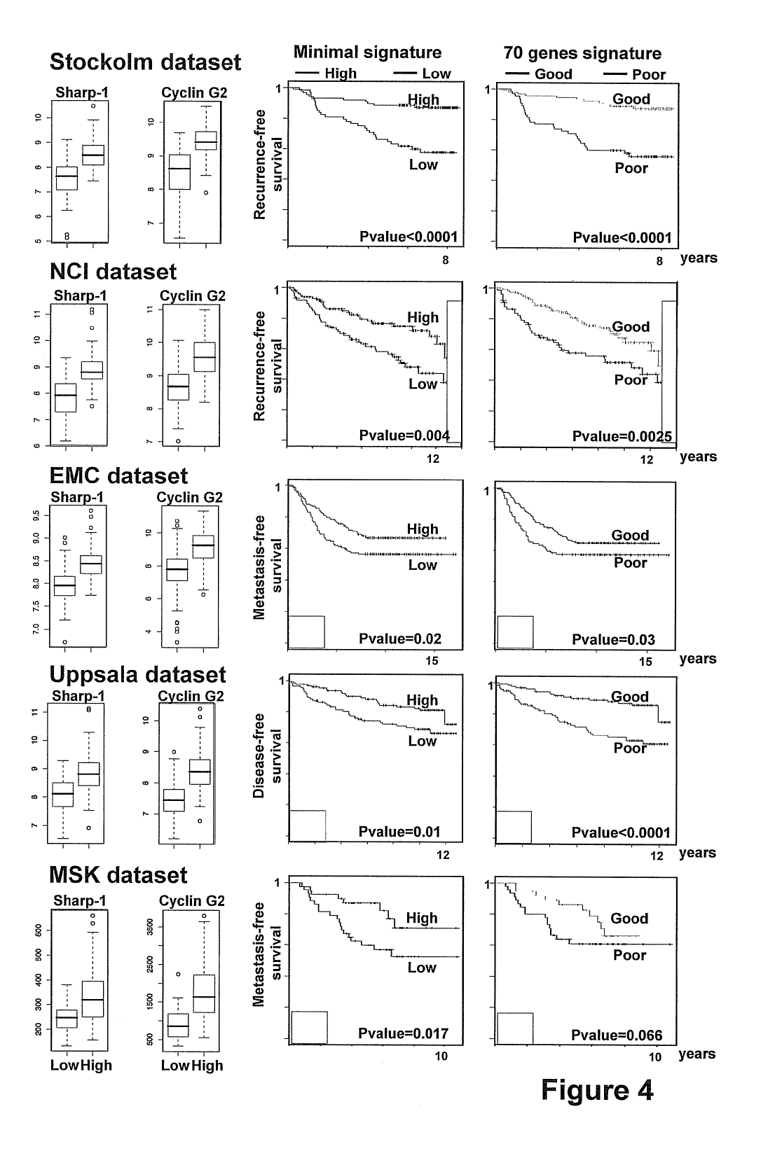

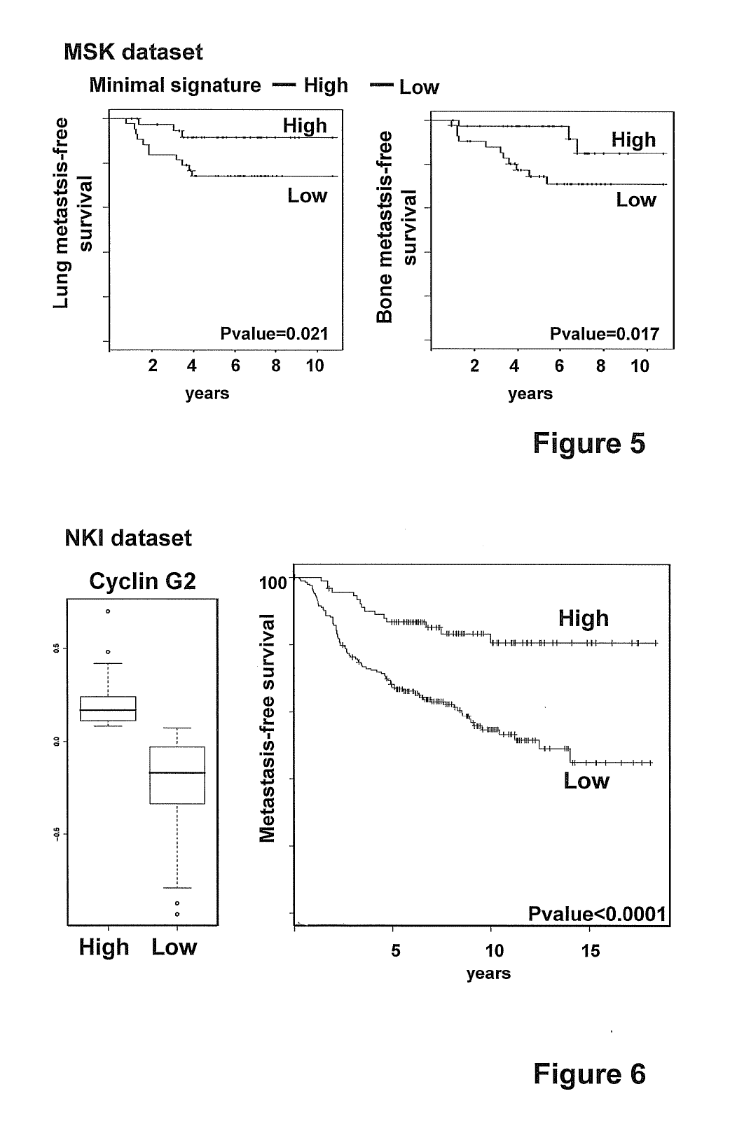

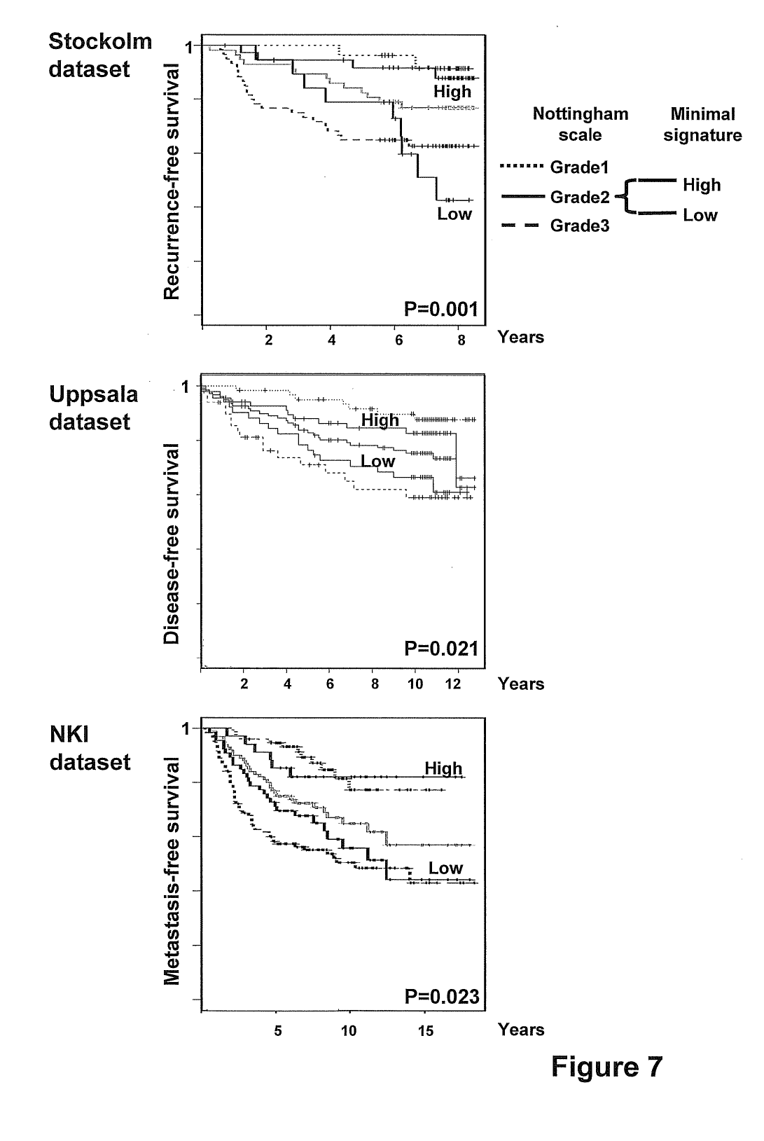

We sought to investigate the effects of mutant-p53 on the cellular response to TGFβ. To this end, we used p53-null H1299 cells stably reconstituted with inducible expression vectors coding for the hot-spot p53R175H mutant allele. This cell line retained similar responsiveness to TGFβ compared to parental H1299, as judged by activation of P-Smad3 (FIG. 1A).

TGFβ treatment of H1299 cells bearing p53R175H caused a strikingly morphology change, as cells shed their cuboidal epithelial shape and acquired a more mesenchymal phenotype, characterized by a number of dynamic protrusions, such as filopodia and lamellipodia (FIG. 1 B). These were not present in parental cells or in cells reconstituted with wild-type p53 (FIG. 1B and data not shown). To examine if expression of mutant-p53 also conferred migratory properties to cells receiving TGFβ, we used a wounding assay, in which cells are induced to disrupt cell-cell contacts, polarize and migrate into a wound created by scratching confluent cultures with a pipette tip. After 30 hours of TGFβ treatment, while parental (p53-null) H1299 cells had migrated poorly, p53R175H expressing cells almost completely invaded the wound (FIG. 1C). To ascribe this effect to cell migration, rather than to a bias in proliferation, we monitored BrdU incorporation and found no difference between TGFβ treated control or mutant-p53 expressing cells (data not shown). As an independent mean of measuring cell motility, we examined the behavior of parental, wild-type or mutant-p53 reconstituted H1299 cells in transwell-migration assays. FIG. 1D shows that expression of mutant-p53, but not of wild-type p53, parallels with the acquisition of a TGFβ pro-migratory response.

These data link the gain of mutant-p53 to TGFβ induced epithelial plasticity and migration, phenotypes whose emergence is critical for TGFβ invasive properties (Gupta and Massague, 2006).

### EXAMPLE 2

### Mutant-p53 and TGFβ Jointly Control Cell Shape and Invasiveness of Breast Cancer Cells in Vitro

To demonstrate the actual requirement for an enhanced epithelial plasticity and migration in metastatic cancer cells with endogenous mutant p53, we stably knocked down endogenous mutant-p53 (p53R280K) in MDA-MB-231 cells, a well-established model of invasive breast cancer (Arteaga et al., 1993; Bandyopadhyay et al., 1999; Deckers et al., 2006; Padua et al., 2008). Cells were transduced with retroviral vectors expressing either shGFP (control), or shRNA targeting p53 (shp53) (see Table 1) and then drug-selected to enrich for positive transfectants. By immunoblotting, expression of shp53 reduced the endogenous level of mutant-p53 protein by >90% (FIG. 2A). In transwell-migration assays, TGFβ triggered a potent promigratory response in control MDA-MB-231 cells. Remarkably, this response was lost in mutant-p53-depleted cells (FIG. 2B). Similar results were obtained upon transient depletion of p53 using two independent anti-p53 siRNA sequences (data not shown). Once embedded in a drop of Matrigel, MDA-MB-231 cells display a TGFβ dependent scattering, extracellular matrix degradation and migration (FIGS. 2C and 2D), recapitulating in vivo invasiveness (Albini, 1998). We found that mutant-p53 expression is required for these activities. These data suggest that, at least in vitro, mutant-p53 and TGFβ jointly control cell shape and invasiveness of breast cancer cells.

### EXAMPLE 3

### Mutant-p53 Expression Plays a Crucial Role in Canalizing TGFβ Responsiveness for Efficient Metastatic Spread in Vivo

Multiple evidences indicate that the metastatic spread of MDA-MB-231 cells in vivo is under control of autocrine TGFβ (Arteaga et al., 1993; Bandyopadhyay et al., 1999; Deckers et al., 2006; Padua et al., 2008). To test if mutant-p53 is relevant for TGFβ promoted malignant behaviors in vivo, we injected shGFP- or shp53-MDA-MB-231 cells into the mammary fat pad of immunocompromized mice. The two cell populations grew at similar rate in vitro (data not shown) and formed primary tumors at similar rates and size in vivo (FIG. 2E), indicating that high levels of mutant-p53 in MDA-MB-231 cells are not essential for proliferation or primary tumor formation. Six weeks after implantation, mice were sacrificed and examined for presence of metastatic lesions.

Orthotopically injected MDA-MB-231 are very poorly metastatic to the lung, but efficiently metastasize to the lymph nodes. To quantify metastatic spread, we monitored the colonization of controlateral lymph nodes, a read-out of systemic disease in human breast cancers (Singletary et al., 2006). Strikingly, suppression of mutant-p53 expression drastically reduced the number of lymph node metastases when compared to the control cells, as only one out of 22 mice injected with the shGFP cells scored negative for lymphonodal metastasis, whereas 10 out of 22 of mice carrying the shp53-depleted tumors remained metastasis-free (FIG. 2F).

To confirm these results implicating mutant-p53 in invasiveness in vivo, we injected control and shp53-MDA-MB-231 intravenously into nude mice. Using two independent clones, we found that depletion of mutant-p53 had a remarkable impact on lung colonization, with overt reduction of metastatic nodules in number and size (FIGS. 2G-2I). Thus, mutant-p53 expression plays a crucial role in canalizing TGFβ responsiveness for efficient metastatic spread.

### EXAMPLE 4

### Identification of the Gene Set Co-Regulated by Mutant-p53 and TGFβ

We next sought to investigate the specific gene expression program by which mutant-p53 and TGFβ control invasion and metastasis. To identify this gene-set, we compared the TGFβ transcriptomic profile of control and mutant-p53 depleted MDA-MB-231 cells. We found that TGFβ potentially regulates more than 400 genes. The large majority of them were expressed independently from the presence of mutant p53.

Among the mutant-p53-independent targets, several had been previously described as direct Smad targets, such as PAI1/SERPINE1, JunB and Smad7 (Massague and Gomis, 2006). Moreover, multiple genes previously associated to a general epithelial “TGFβ response classifier” were also found, including genes associated to lung or bone specific metastasis (ANGPTL4, NEDD9, IL11 and CTGF) (Padua et al., 2008). The successful identification of these targets validated our procedure to identify novel genes that may play important roles in TGFβ induced malignancy. Interestingly, we highlighted 147 genes previously implicated in cell movement, invasion or metastasis (FIG. 3A and data not shown).

However, TGFβ needs the presence of mutant p53 to exploit its pro-metastatic function; we therefore restricted our attention to a much smaller set of genes co-regulated by mutant-p53 and TGFβ; strikingly, this entailed only five genes: Sharp1/DEC2/BHLHB3/BHLHE41, CyclinG2/CCNG2, ADAMTS9, Follistatin and GPR87 (see FIGS. 3B and 3C). In particular, we focused on two candidate metastasis suppressors, Sharp1 and CyclinG2, that are negatively regulated by TGFβ via mutant-p53 (FIG. 3D). Sharp1 is an inhibitory basic helix-loop-helix resembling ID-proteins (i.e. in MyoD inhibition assays) (Li et al., 2003), but whose biological roles are otherwise largely unknown. CyclinG2 is considered an atypical “inhibitory” cyclin, but can also influence the dynamic of the microtubule cytoskeleton; intriguingly, CyclinG2 is asymmetrically inherited during cell division, in virtue of its association with the centrosome surrounding the mother centriole (Arachchige Don et al., 2006).

### EXAMPLE 5

### Biological Validation of the Identified Gene Set in Vitro

To functionally validate these genes as effectors of the mutant-p53/TGFβ pathway, we carried out epistasis experiments testing if depletion of Sharp1 or CyclinG2 could rescue TGFβ induced migration in p53-depleted cells. As shown in FIG. 3E, siRNA-mediated knockdowns of Sharp1 or CyclinG2 restore TGFβ dependent pro-migratory activities in shp53 MDA-MB-231 (FIG. 3E, compare lanes 3 and 4 with lane 2) Thus, these molecules antagonize TGFβ proinvasive responses, acting as metastasis suppressors. Having identified genes essential to antagonize invasive behaviour in vitro, we then sought to elucidate their clinical relevance as metastasis suppressors. Recent transcriptomic profilings of primary human tumors have identified gene suites, or “signatures”, that predict high risk of metastasis and poor disease-free survival (Fan et al., 2006; van't Veer et al., 2002). If the detection of Sharp1 and CyclinG2 in primary tumors is biologically meaningful, one might expect that reduced expression of these genes should be associated with poor clinical outcome. Surprisingly, Sharp1 and CyclinG2 are not contained in known signatures for breast cancer metastasis, i.e. the 70-genes signature, the recurrence score or others (Fan et al., 2006).

### EXAMPLE 6

### Prognostic Validation of the gene set Identified by Statistical Analysis and Comparison with Other Gene Sets

Breast Cancer Dataset

To evaluate the prognostic value of Sharp1 and CyclinG2, we collected 6 different datasets (Table 3). For each data set, we performed survival analysis to test if the minimal signature could classify patients into clinically distinct groups. Each dataset has been processed independently from the other to preserve the original differences among the various studies (e.g., patient cohort, microarray type, sample processing protocol, etc.).

To evaluate the prognostic value of Sharp1 and CyclinG2 (Minimal Signature, MS), we took advantage of the available gene expression datasets summing up to 900 primary breast cancers with associated clinical data, including survival and distant recurrence.

TABLE 3
Breast cancer datasets analyzed in this study

| | Microarray | | | |
|---|---|---|---|---|
| Study | platform | Samples | Data source | Reference |
| Stockholm | Affymetrix | 156 | GEO GSE1456 | (Pawitan et al., |
| | HG-U133A | | | 2005) |
| NCI | Affymetrix | 187 | GEO GSE2990 | (Sotiriou et al., |
| | HG-U133A | | | 2006) |
| EMC | Affymetrix | 286 | GEO GSE2034 | (Wang et al., |
| | HG-U133A | | | 1998) |
| Uppsala | Affymetrix | 236 | GEO GSE3494 | (Miller et al., |
| | HG-U133A | | | 2005) |
| MSK | Affymetrix | 82 | GEO GSE2603 | (Minn et al., |
| | HG-U133 | | | 2005) |
| NKI | Agilent, | 295 | http://www.rii.com/ | (Fan et al., 2006; |
| | Rosetta | | publications/2002/ | van't Veer et al., |
| | Inpharmatics | | nejm.html; | 2002; van de |
| | | | http://microarray- | Vijver et al., |
| | | | pubs.stanford.edu/ | 2002) |
| | | | wound_NKI/ | |
| | | | explore.html | |

We downloaded breast cancer gene expression datasets with clinical information from Gene Expression Omnibus (http://www.ncbi.nlm.nih.gov/GEO/), Stanford Microarray Database (http://genome-www5.stanford.edu/), or author's individual web pages (http://microarray-pubs.stanford.edu/wound_NKI/explore.html).

Table 3 reports the complete list of datasets and their sources. With the exception of EMC, MSK and NKI studies, raw data (e.g., CEL files) were available for all samples. Detailed clinical information could be acquired for any analyzed sample. The datasets included both Affymetrix and dual-channel cDNA microarray platforms. Since all Affymetrix data were from the same HG-U133A platform, no method was needed to map probesets across various generations of Affymetrix GeneChip arrays. When CEL files were available, expression values were generated from intensity signals using the RMA algorithm; values have been background adjusted, normalized using quantile normalization, and expression measure calculated using median polish summarization. In the case of EMC, MSK and NKI studies, data were used as downloaded. Specifically, in the EMC and MSK datasets expression values were calculated using Affymetrix MAS 5.0 algorithm. In Affymetrix HG-U133A array, CyclinG2 is represented by 3 probesets (202769_at, 202770_s_at, and 211559_s_at), while Sharp1 is interrogated only by probeset 221530_s_at.

The Agilent, Rosetta Inpharmatics array used for the NKI dataset has a single probe for CyclinG2 while does not contain any probe for Sharp1.

Minimal Signature Classification

To identify two groups of samples with either high or low simultaneous expression scores of Sharp1 and CyclinG2, we defined a classification rule based on summarizing the standardized expression levels of Sharp1 and CyclinG2 into a combined score with zero mean.

Tumors were then classified as minimal signature Low if the combined score is negative and as minimal signature High if the combined score is positive:

$$minimal signature Low -> \frac{{x}_{i}^{Sharp - 1} - {\hat{μ}}^{Sharp - 1}}{{\hat{σ}}^{Sharp - 1}} + \frac{{x}_{i}^{CyclinG 2} - {\hat{μ}}^{CyclinG 2}}{{\hat{σ}}^{Cyclin G 2}} \leq 0$$

$$minimal signature High -> \frac{{x}_{i}^{Sharp - 1} - {\hat{μ}}^{Sharp - 1}}{{\hat{σ}}^{Sharp - 1}} + \frac{{x}_{i}^{CyclinG 2} - {\hat{μ}}^{Cyclin G 2}}{{\hat{σ}}^{CyclinG 2}} > 0$$

where xᵢ^{Sharp-1}, xᵢ^{CyclinG2} are the expression levels of Sharp1 and CyclinG2 in sample i and \hat{μ}^{Sharp-1}, \hat{μ}^{CyclinG2}, \hat{σ}^{Sharp-1} and \hat{σ}^{CyclinG2} are the estimated means and standard deviations of Sharp1 and CyclinG2 calculated over the entire dataset.

This classification was applied for Stockholm, NCI and Uppsala studies based on expression values obtained from RMA, whereas for EMC and MSK expression values have been used as downloaded. In the case of EMC dataset, expression data have been log 2-transformed.

In the case of the NKI dataset, samples had to be classified in High and Low groups based on CyclinG2 data only.

To determine the appropriate threshold of CyclinG2 expression level, we used the clinical parameters to quantify the proportion of patients with good clinical outcome, i.e. lymph node negative patients who remained free of metastases after at least 5 years of follow-up (van't Veer et al., 2002). Since about 31% of the samples met these criteria (92 out of 295 tumors), the 69^{th} percentile of CyclinG2 is expression values (i.e. 0.078) was used as the cut-off to classified tumors in either High or Low groups: if CyclinG2 expression level of a given sample was higher than the 69^{th} percentile of CyclinG2 values, then the sample was termed minimal signature High, otherwise, it was termed minimal signature Low. The rationale behind this choice is that about 31% of the patients were expected to be classified as minimal signature High.

Samples were also classified into the minimal signature High and minimal signature Low groups based on the expression levels of Sharp1 and CyclinG2 using unsupervised clustering techniques (Pollard, 2005).

In particular, agglomerative clustering with Euclidean distance and complete or Ward's linkage criteria has been used for the classification of MSK and EMC datasets, respectively; divisive clustering with Euclidean distance (diana) has been applied to the NCI samples and the k-means partitioning algorithm has been used for the Stockholm and Uppsala datasets. The clustering methods were not applied to the NKI samples as gene expression data are available only for CyclinG2.

We compared the performance of the minimal signature and of the 70-genes signature for all the analyzed dataset. Since all dataset other than NKI are from Affymetrix arrays, we first mapped genes of the 70-genes signature to Affymetrix probesets, obtaining that the NKI 70-gene poor prognosis signature maps to 75 probesets in the Affymetrix U133A platform corresponding to 48 unique EntrezGene IDs. Given this reduction on the number of genes making up the signature and given the fact that we used a different model for classifying patients, we verified if the prognostic performance of a different model (i.e., an unsupervised clustering) constructed on a reduced gene list is similar to that of van't Veer's model based on the full signature. Thus, we classified NKI samples using the 48 unique genes that are present on both Affymetrix and Rosetta platforms and a classification model based on unsupervised clustering. In agreement to what previously reported by van't Veer et al., 2002 and by Minn et al., 2005, we found that using an unsupervised clustering on a reduced signature had little impact on the performance of the classifier. Thus, samples in all other data sets have been classified into two groups using this reduced 70-gene signature and unsupervised clustering. In particular, an agglomerative hierarchical is model based on Ward's algorithm (Ward, 1963) was used for the Stockholm study, the Uppsala and ECM studies were classified using PAM algorithm (Kaufman and Rousseeuw, 1990). Finally, for MSK study, we used the classification given by Minn et al, 2005.

Survival Analysis

To evaluate the prognostic value of the minimal signature, we estimated, using the Kaplan-Meier method (Prentice, 1978), the probabilities that patients would remain free of metastases (MSK and NKI), free of tumor recurrence (Stockholm and NCI), and free of cancer disease (Uppsala) according to whether they belong to High or Low group. To confirm these findings, the survival curves were compared using the log-rank or Mantel-Haenszel test (Harrington and Fleming, 1982), i.e. testing the null hypothesis of no difference against the one-sided alternative supporting minimal signature High survival. P-values were calculated according to the standard normal asymptotic distribution and adjusted according to sequential Bonferroni-Holm multiple test procedure (Dudoit, 2003) to control the family-wise error rate. All the adjusted p-values were significant at a level a=0.05 when comparing minimal signature High and minimal signature Low groups as defined using the combined score. The same survival analysis repeated on minimal signature High and minimal signature Low groups as defined using the clustering techniques returned similar results, with p-values of Stockholm: 0.00026, NCI: 0.00083, EMC: 0.0251, Uppsala: 0.0025, MSK: 0.00887.

Finally, the survival analysis was applied to subsets of samples assigned to High and Low groups and classified as intermediate (grade 2) by the Nottingham scale. Again, all null hypotheses was rejected controlling the family-wise error rate at a=0.05. In the case of the NCI dataset, this analysis could not be performed since the recurrence-free survival curve for grade 2 tumors is not statistically different from the curve of poorly differentiated grade 3 tumors. Information for the Nottingham scale classification of the tumors is not available in the MSK and EMC datasets.

Conclusion

After having defined in each dataset two groups of tumors with respectively high and low level of expression of Sharp1 and CyclinG2 (FIG. 4), it was found that, strikingly, the group expressing low levels of the minimal signature displayed a significant higher probability to develop recurrence when compared to the “High” group (p-values ranged from 0.02 to 3E-05, depending on the datasets) when tested using the univariate Kaplan-Meier survival analysis.

Interestingly, the MS performed comparably to the 70-genes profile, in stratifying patients according to their clinical outcome (FIG. 4).

The expressions of Sharp1 and CyclinG2 are synergic for the predictive power of the minimal signature in these assays and are associated to risk of distant metastasis to both bone and lung (FIG. 5). That said, in patient datasets for which Sharp1 expression data were not available, such as the NKI dataset (295 tumors) (Fan et al., 2006), the stratification based on the sole CyclinG2 remains predictive of metastasis (see FIG. 6).

Multivariate Analysis Using a Cox Proportional-Hazards Model

To further evaluate the prognostic value of the minimal signature we performed multivariate Cox proportional-hazards analysis on the 187 tumors dataset from National Cancer Institute (Sotiriou et al., 2006). In particular, it was examined the risk of recurrence for the 187 tumors from the NCI study by the Cox proportional-hazards regression modeling (Cox, 1972).

The relationship between survival and the minimal signature predictor and other predictors commonly used in the clinical practice, including tumor diameter, estrogen-receptor status (ER positive vs. negative), nodal status (positive vs. negative), tumor grade (grade 2 vs. grade 1 and grade 3 vs. grade 1) and treatment status (tamoxifen vs. none) was specifically examined.

We fitted Cox proportional-hazards regression model first by using clinical variables only (Model 1), and then adding the minimal signature predictor (Model 2). Results are given in Tables 4 and 5 showing that the Minimal Signature remained a significant predictor of metastasis-free survival thus adding new prognostic information beyond that one provided by the standard clinical predictors.

TABLE 4
Multivariate analysis of the risk of recurrence for the NCI dataset
using a Cox proportional-hazards model
In Model 1, tumor size and grade 2 (versus grade 1) covariates have
statistically significant coefficients at α = 0.05. However, when the minimal
signature is included (Model 2), affiliation to group ‘Low’, keeping constant
all other covariates, significantly increases the hazard of recurrence by a
factor of e^{0.706} = 2.026 on average, i.e. adds new prognostic information.
Model 1: Multivariate analysis using clinical variables only.
Model 1 was obtained using n = 159 observations and its, residual deviance
(i.e., minus twice the partial log likelihood) is equal to RD1= 492.8774

| | Hazard | Hazard ratio 95% | |
|---|---|---|---|
| Variable | ratio | confidence interval | p-value |
| Tumor diameter >2 cm (<= | 2.206 | (1.242-3.92) | 0.0069 |
| 2 cm) | | | |
| Node positive (vs. node | 0.815 | (0.304-2.19) | 0.6900 |
| negative) | | | |
| Grade 2 (vs. Grade 1) | 2.327 | (1.037-5.22) | 0.0410 |
| Grade 3 (vs. Grade 1) | 1.282 | (0.597-2.75) | 0.5200 |
| ER positive (vs. ER negative) | 0.790 | (0.414-1.50) | 0.4700 |
| Tamoxifen treatment | 1.564 | (0.645-3.79) | 0.3200 |

Model 2: Multivariate analysis using clinical variables and
the minimal signature.
Model 2 was obtained using n = 159 observations and its residual deviance
(i.e., minus twice the partial log likelihood) is equal to RD2 = 486.8369.

| | Hazard | Hazard ratio 95% | |
|---|---|---|---|
| Variable | ratio | confidence interval | p-value |
| Tumor size (cm) | 2.198 | (1.228-3.94) | 0.008 |
| Node positive (vs. node | 0.787 | (0.294-2.11) | 0.630 |
| negative) | | | |
| Grade 2 (vs. Grade 1) | 2.084 | (0.927-4.68) | 0.076 |
| Grade 3 (vs. Grade 1) | 0.973 | (0.437-2.17) | 0.950 |
| ER positive (vs. ER | | | |
| negative) | 0.818 | (0.427-1.57) | 0.540 |
| Tamoxifen treatment | 1.504 | (0.618-3.66) | 0.370 |
| Group Low (vs. Group High) | 2.026 | (1.141-3.60) | 0.016 |

Model 1 and Model 2 may be compared to assess whether the minimal signature adds additional prognostic information over the clinical variables. In particular, this is obtained by subtracting the residual deviance of Model 1 (RD1=492.8774) from the one of Model 2 (RD2=486.8369) and testing this difference (RD1−RD2=6.04043) against a chi-square distribution with one degree of freedom. Since this difference exceeds the 0.95 quantile of the chi-square distribution with one degree of freedom (p-value=0.01398) the minimal signature is a significant predictor of recurrence-free survival, adding new prognostic information beyond the one provided by the standard clinical predictors.

TABLE 5
Statistical comparison between models obtained using single clinical
variables and models obtained adding the minimal signature.

| Clinical | Difference of residual | |
|---|---|---|
| predictor | deviances | p-value |
| Tumor size | 4.3611 | 0.0368 |
| Nodal status | 7.4596 | 0.0063 |
| Tumor grade | 5.6859 | 0.0171 |
| ER status | 6.6992 | 0.0096 |
| Treatment status | 6.772 | 0.0093 |

In addition, the minimal signature adds prognostic value not only to the multivariate model but also to any model constructed using any single clinical predictor. Indeed, the difference between the residual deviance of the model obtained using a single clinical variable plus the minimal signature (e.g. tumor diameter+minimal signature) and the residual deviance of the model obtained using only a clinical variable, is significant for each clinical predictor.

The above provided data confirm that the present invention provides additional prognostic tools for assessing the risk of metastasis, thus identifying patients that would benefit from adjuvant treatments.

Moreover, a point in case are tumors classified as intermediate (grade 2) by the Nottingham scale, that represent the majority of tumors and whose prognosis is uncertain (Ivshina et al., 2006). When applied to grade 2 tumors of multiple independent datasets, the minimal signature resolved these patients into two is groups with outcomes comparable to grade 1 and grade 3, respectively (FIG. 7). This result has not been achieved by any other, even more complex molecular method, thus being peculiar to the present invention.

### REFERENCES

Albini, A. (1998). Tumor and endothelial cell invasion of basement membranes. The matrigel chemoinvasion assay as a tool for dissecting molecular mechanisms. Pathol Oncol Res 4, 230-241.

Arachchige Don, A. S., Dallapiazza, R. F., Bennin, D. A., Brake, T., Cowan, C. E., and Horne, M. C. (2006). CyclinG2 is a centrosome-associated nucleocytoplasmic shuttling protein that influences microtubule stability and induces a p53-dependent cell cycle arrest. Experimental cell research 312, 4181-4204.

Arteaga, C. L., Hurd, S. D., Winnier, A. R., Johnson, M. D., Fendly, B. M., and Forbes, J. T. (1993). Anti-transforming growth factor (TGF)-beta antibodies inhibit breast cancer cell tumorigenicity and increase mouse spleen natural killer cell activity. Implications for a possible role of tumor cell/host TGF-beta interactions in human breast cancer progression. The Journal of clinical investigation 92, 2569-2576.

Bandyopadhyay, A., Zhu, Y., Cibull, M. L., Bao, L., Chen, C., and Sun, L. (1999). A soluble transforming growth factor beta type III receptor suppresses tumorigenicity and metastasis of human breast cancer MDA-MB-231 cells. Cancer research 59, 5041-5046.

Beenken, S. W., Grizzle, W. E., Crowe, D. R., Conner, M. G., Weiss, H. L., Sellers, M. T., Krontiras, H., Urist, M. M., and Bland, K. I. (2001). Molecular biomarkers for breast cancer prognosis: coexpression of c-erbB-2 and p53. Annals of surgery 233, 630-638.

Cordenonsi, M., Dupont, S., Maretto, S., Insinga, A., Imbriano, C., and Piccolo, S. (2003). Links between tumor suppressors: p53 is required for TGF-beta gene responses by cooperating with Smads. Cell 113, 301-314.

Cox, D. R. (1972). Regression Models and Life Tables (with Discussion). Journal of the Royal Statistical Society, Series B-Statistical Methodology 34, 34.

Deckers, M., van Dinther, M., Buijs, J., Que, I., Lowik, C., van der Pluijm, G., and ten Dijke, P. (2006). The tumor suppressor Smad4 is required for transforming growth factor beta-induced epithelial to mesenchymal transition and bone metastasis of breast cancer cells. Cancer research 66, 2202-2209.

Dudoit, S., Popper Shaffer. J., Boldrick, J. C. (2003). Multiple Hypothesis Testing in Microarray Experiments. Statistical Science 18, 71-103.

Fan, C., Oh, D. S., Wessels, L., Weigelt, B., Nuyten, D. S., Nobel, A. B., van't Veer, L. J., and Perou, C. M. (2006). Concordance among gene-expression-based predictors for breast cancer. The New England journal of medicine 355, 560-569.

Gupta, G. P., and Massague, J. (2006). Cancer metastasis: building a framework. Cell 127, 679-695.

Harrington, D. P., and Fleming, T. R. (1982). A class of rank test procedures for censored survival data. Biometrika 69, 4.

t Hoen, P. A., Ariyurek, Y., Thygesen, H. H., Vreugdenhil, E., Vossen, R. H., de Menezes, R. X., Boer, J. M., van Ommen, G. J., and den Dunnen, J. T. (2008). Deep sequencing-based expression analysis shows major advances in robustness, resolution and inter-lab portability over five microarray platforms. Nucleic acids research 36, e141.

Hartigan, J. A., and Wong, M. A. (1979). A K-means clustering algorithm. Applied Statistics 28, 9.

Irizarry, R. A., Bolstad, B. M., Collin, F., Cope, L. M., Hobbs, B., and Speed, T. P. (2003). Summaries of Affymetrix GeneChip probe level data. Nucleic Acids Res 31, e15.

Ivshina, A. V., George, J., Senko, O., Mow, B., Putti, T. C., Smeds, J., Lindahl, T., Pawitan, Y., Hall, P., Nordgren, H., et al. (2006). Genetic reclassification of histologic grade delineates new clinical subtypes of breast cancer. Cancer research 66, 10292-10301.

Li, Y., Xie, M., Song, X., Gragen, S., Sachdeva, K., Wan, Y., and Yan, B. (2003). DEC1 negatively regulates the expression of DEC2 through binding to the E-box in the proximal promoter. The Journal of biological chemistry 278, 16899-16907.

Miki, Y., Swensen, J., Shattuck-Eidens, D., Futreal, P. A., Harshman, K., Tavtigian, S., Liu, Q., Cochran, C., Bennett, L. M., Ding, W., et al. (1994). A strong candidate for the breast and ovarian cancer susceptibility gene BRCA1. Science (New York, N.Y. 266, 66-71.

Miller, L. D., Smeds, J., George, J., Vega, V. B., Vergara, L., Ploner, A., Pawitan, Y., Hall, P., Klaar, S., Liu, E. T., et al. (2005). An expression signature for p53 status in human breast cancer predicts mutation status, transcriptional effects, and patient survival. Proceedings of the National Academy of Sciences of the United States of America 102, 13550-13555.

Minn, A. J., Gupta, G. P., Siegel, P. M., Bos, P. D., Shu, W., Giri, D. D., Viale, A., Olshen, A. B., Gerald, W. L., and Massague, J. (2005). Genes that mediate breast cancer metastasis to lung. Nature 436, 518-524.

Padua, D., Zhang, X. H., Wang, Q., Nadal, C., Gerald, W. L., Gomis, R. R., and Massague, J. (2008). TGFbeta primes breast tumors for lung metastasis seeding through angiopoietin-like 4. Cell 133, 66-77.

Pawitan, Y., Bjohle, J., Amler, L., Borg, A. L., Egyhazi, S., Hall, P., Han, X., Holmberg, L., Huang, F., Klaar, S., et al. (2005). Gene expression profiling spares early breast cancer patients from adjuvant therapy: derived and validated in two population-based cohorts. Breast Cancer Res 7, R953-964.

Piccolo, S., Agius, E., Leyns, L., Bhattacharyya, S., Grunz, H., Bouwmeester, T., and De Robertis, E. M. (1999). The head inducer Cerberus is a multifunctional antagonist of Nodal, BMP and Wnt signals. Nature 397, 707-710.

Pollard, K. S., van der Laan, M. J. (2005). Cluster Analysis of Genomic Data with Applications in R. U.C. Berkeley Division of Biostatistics Working Paper Series Working Paper 167.

Prentice, R. L., Gloeckler, L. A. (1978). Regression Analysis of Grouped Survival Data with Application to Breast Cancer Data. Biometrics 34, 57-67.

Singletary, S. E., and Connolly, J. L. (2006). Breast cancer staging: working with the sixth edition of the AJCC Cancer Staging Manual. CA: a cancer journal for clinicians 56, 37-47.

Sotiriou, C., Wirapati, P., Loi, S., Harris, A., Fox, S., Smeds, J., Nordgren, H., Farmer, P., Praz, V., Haibe-Kains, B., et al. (2006). Gene expression profiling in breast cancer: understanding the molecular basis of histologic grade to improve prognosis. Journal of the National Cancer Institute 98, 262-272.

Storey, J. D. (2002). A direct approach to false discovery rates. Journal of the Royal Statistical Society Series B-Statistical Methodology 64, 479-498.

Tusher, V. G., Tibshirani, R., and Chu, G. (2001). Significance analysis of microarrays applied to the ionizing radiation response. Proc Natl Acad Sci USA 98, 5116-5121.

van't Veer, L. J., Dai, H., van de Vijver, M. J., He, Y. D., Hart, A. A., Mao, M., Peterse, H. L., van der Kooy, K., Marton, M. J., Witteveen, A. T., et al. (2002). Gene expression profiling predicts clinical outcome of breast cancer. Nature 415, 530-536.

van de Vijver, M. J., He, Y. D., van't Veer, L. J., Dai, H., Hart, A. A., Voskuil, D. W., Schreiber, G. J., Peterse, J. L., Roberts, C., Marton, M. J., et al. (2002). A gene-expression signature as a predictor of survival in breast cancer. The New England journal of medicine 347, 1999-2009.

Wang, X. J., Greenhalgh, D. A., Jiang, A., He, D., Zhong, L., Medina, D., Brinkley, B. R., and Roop, D. R. (1998). Expression of a p53 mutant in the epidermis of transgenic mice accelerates chemical carcinogenesis. Oncogene 17, 35-45.

Ward, J. H. (1963). Hierarchical Grouping to optimize an objective function. Journal of American Statistical Association 301, 9.

1. A method to evaluate a breast cancer patient's risk of recurrence comprising detecting the level of CyclinG2 (Gene ID=901) gene expression alone or in combination with Sharp1 (Gene ID=79365) in a sample.

2. The method according to claim 1 wherein said detection comprises measuring a signal and acquiring it.

3. The method according to claims 1-2 further comprising the following step:
calculating a signature score for CyclinG2 alone or for, preferably, both CyclinG2 and Sharp1 in the unknown sample, wherein said signature score is defined as:
$${\sum }_{k = 1}^{K} \frac{{x}_{i}^{k} - {\hat{μ}}^{k}}{{\hat{σ}}^{k}}$$
being K=1 when using CyclinG2 alone and K=2 when using both CyclinG2 and Sharp1, xᵢ^{k} the expression level of CyclinG2 or Sharp1 in the unknown sample i, \hat{μ}^{k} and \hat{σ}^{k} respectively the estimated mean and standard deviation values of the CyclinG2 alone or in combination with Sharp1 expression levels in a breast cancer patients population with known clinical history,
wherein a signature score lower than zero or equal to zero indicates an increased risk of breast cancer recurrence.

4. The method according to any one of claims 1-3, wherein said detection is carried out by molecular and/or immunological means.

5. The method according to any ones of claims 1-4 wherein said molecular means are selected from the group consisting of: PCR, microarray analysis, deep sequencing, Northern-blot.

6. The method according to claim 5 wherein said PCR is a Real Time PCR or a Quantitative PCR.

7. The method according to any one of claims 1-6 wherein said sample is a breast cancer biopsy or a nucleic acid isolated from said breast cancer biopsy.

8. A method according to any one of claims 2-7 further comprising the following steps:
quality control of the acquired signal,
normalization of the signal;
optional rescaling of the signal.

9. The method according to any one of claims 3-8 further comprising the following steps:
i) defining a minimal signature template consisting in the mean and standard deviations of Sharp1 and CyclinG2, \hat{μ}^{Sharp-1}, \hat{μ}^{CyclinG2}, \hat{σ}^{Sharp-1} and \hat{σ}^{CyclinG2} expression values in a population of samples with known clinical history;
ii) calculating a signature score as defined in claim 3 for CyclinG2 or for CyclinG2 and Sharp1 gene expression in the unknown sample;
iii) classifying the unknown sample in the minimal signature Low group when its signature score is negative or in the minimal signature High when its signature score is positive, according to the following calculation:
$$minimal signature Low -> \frac{{x}_{i}^{Sharp - 1} - {\hat{μ}}^{Sharp - 1}}{{\hat{σ}}^{Sharp - 1}} + \frac{{x}_{i}^{CyclinG 2} - {\hat{μ}}^{Cyclin G 2}}{{\hat{σ}}^{CyclinG 2}} \leq 0$$
$$minimal signature High -> \frac{{x}_{i}^{Sharp - 1} - {\hat{μ}}^{Sharp - 1}}{{\hat{σ}}^{Sharp - 1}} + \frac{{x}_{i}^{CyclinG 2} - {\hat{μ}}^{Cyclin G 2}}{{\hat{σ}}^{CyclinG 2}} > 0$$
wherein xᵢ^{Sharp-1} and xᵢ^{CyclinG2} are the expression levels of Sharp1 and CyclinG2 in the unknown sample and \hat{μ}^{Sharp-1}, \hat{μ}^{CyclinG2}, \hat{σ}^{Sharp-1} and \hat{σ}^{CyclinG2} are the estimated means and standard deviations of Sharp1 and CyclinG2 calculated over a dataset composed of samples with known clinical history, and
wherein classification into the minimal signature Low group is an indication of an high risk of cancer recurrence for a breast cancer patient.

10. The method according to claims 8-9 wherein at least the steps of:
signal acquisition
quality control of the acquired signal,
normalization of the acquired signal;
are carried out by software run on a computer.

11. The method according to claim 10 wherein also steps i-iii) as defined in claim 9 are carried out by software run on a computer.

12. A method for analysing a breast cancer dataset comprising CyclinG2 and/or Sharp1 gene expression data, comprising the calculation of a minimal signature template as defined in claim 9 i) for CyclinG2 and preferably also for Sharp1 gene expression data.

13. Use of CyclinG2 (Gene ID=901) gene expression for evaluating a breast cancer patient's risk of cancer recurrence.

14. The use according to claim 13 further comprising the evaluation of Sharp1 gene expression (Gene ID=79365).

15. The use according to claims 13-14 for further resolution of breast tumors classified as intermediate (grade 2) according to the Nottingham scale.

16. The use according to claims 13-15 wherein said CyclinG2 gene expression is measured with a detecting reagent selected from the group consisting of:
i) CyclinG2-specific oligonucleotide, consisting in an oligonucleotide comprising at least a 13-mer oligonucleotide derived from SEQIDNO:1 or its complementary sequence;
ii) an anti-CyclinG2 specific antibody.

17. The use according to claims 14-16 wherein said Sharp 1 gene expression is measured with a detecting reagent selected from the group consisting of:
i) Sharp1 specific oligonucleotide, consisting in an oligonucleotide comprising at least a 13-mer oligonucleotide derived from SEQIDNO:2 or its complementary sequence;
ii) an anti-Sharp1 specific antibody.

18. A kit for evaluating the expression of CyclinG2 alone or in combination with Sharp1 and determining the risk of cancer recurrence in a sample from a breast cancer patient, comprising:
a CyclinG2-specific reagent, preferably an oligonucleotide consisting in a oligonucleotide comprising at least a 13-mer oligonucleotide derived from SEQIDNO:1 or its complementary sequence;
a Sharp1-specific reagent, preferably an oligonucleotide consisting in an oligonucleotide comprising at least a 13-mer oligonucleotide derived from SEQIDNO:2 or its complementary sequence;
instruction for calculating the signature score of the unknown sample and classifying the unknown sample in the minimal signature Low group when its signature score is negative or in the minimal signature High when its signature score is positive, according to calculation defined in claim 9 i)-iii);
wherein classification into the minimal signature Low group is an indication of an high risk of cancer recurrence for a breast cancer patient.

19. The kit according to claim 18 wherein said instruction are comprised in a software.

20. The kit according to claims 18-19 further comprising as reference standard CyclinG2 and Sharp1 standard expression controls High and Low, expression values or nucleic acid samples.

21. The kit according to claim 20 wherein said expression values or nucleic acid samples are from a non metastatic breast cancer cell line and/or from a highly metastatic cell line.
